(19) United States
(12) Patent Application Publication
(10) Pub. No.: US 2016/0230270 A1
(43) Pub. Date: Aug. 11, 2016

(54) TEMPERATURE-DEPENDENT FABRICATION OF INTEGRATED COMPUTATIONAL ELEMENTS

(71) Applicant: Halliburton Energy Services, Inc., Houston, TX (US)

(72) Inventors: David L. PERKINS, The Woodlands, TX (US); Robert Paul FREESE, Pittsboro, NC (US); Christopher Michael JONES, Houston, TX (US); Richard Neal GARDNER, Raleigh,, NC (US)

(73) Assignee: HALLIBURTON ENERGY SERVICES, INC., Houston, TX (US)

(21) Appl. No.: 14/390,646

(22) PCT Filed: Dec. 30, 2013
(86) PCT No.: PCT/US2013/078365

§ 371 Date: Oct. 3, 2014

(51) Int. Cl. C23C 14/54; G01N 21/84; G01N 21/31; G01N 21/33; E21B 49/08; C23C 14/50; G01N 33/28

(52) U.S. Cl. CPC C23C 14/547; C23C 14/541; C23C 14/545; C23C 14/50; G01N 21/84; G01N 33/2823; G01N 21/33; E21B 49/08; G01N 21/8422; G01N 21/31; G01N 2021/8411; G01N 2021/8438

(57) ABSTRACT

Technologies are described for controlling temperature of ICEs during ICE fabrication. In one aspect, a method includes receiving a design of an integrated computational element (ICE), the ICE design including specification of a substrate and a plurality of layers, their respective target thicknesses and complex refractive indices, where complex refractive indices of adjacent layers are different from each other, and where a notional ICE fabricated in accordance with the ICE design is related to a characteristic of a sample; forming at least some of the plurality of layers of an ICE in accordance with the ICE design; and controlling, during the forming, a temperature of the formed layers of the ICE such that the ICE, when completed, relates to the characteristic of the sample.

### BACKGROUND

The subject matter of this disclosure is generally related to fabrication of an integrated computational element (ICE) used in optical analysis tools for analyzing a substance of interest, for example, crude petroleum, gas, water, or other wellbore fluids. For instance, the disclosed ICE fabrication includes controlling a temperature of the ICEs being fabricated.

Information about a substance can be derived through the interaction of light with that substance. The interaction changes characteristics of the light, for instance the frequency (and corresponding wavelength), intensity, polarization, and/or direction (e.g., through scattering, absorption, reflection or refraction). Chemical, thermal, physical, mechanical, optical or various other characteristics of the substance can be determined based on the changes in the characteristics of the light interacting with the substance. As such, in certain applications, one or more characteristics of crude petroleum, gas, water, or other wellbore fluids can be derived in-situ, e.g., downhole at well sites, as a result of the interaction between these substances and light.

Integrated computational elements (ICEs) enable the measurement of various chemical or physical characteristics through the use of regression techniques. An ICE selectively weights, when operated as part of optical analysis tools, light modified by a sample in at least a portion of a wavelength range such that the weightings are related to one or more characteristics of the sample. An ICE can be an optical substrate with multiple stacked dielectric layers (e.g., from about 2 to about 50 layers), each having a different complex refractive index from its adjacent layers. The specific number of layers, N, the optical properties (e.g. real and imaginary components of complex indices of refraction) of the layers, the optical properties of the substrate, and the physical thickness of each of the layers that compose the ICE are selected so that the light processed by the ICE is related to one or more characteristics of the sample. Because ICEs extract information from the light modified by a sample passively, they can be incorporated in low cost and rugged optical analysis tools. Hence, ICE-based downhole optical analysis tools can provide a relatively low cost, rugged and accurate system for monitoring quality of wellbore fluids, for instance.

Errors in fabrication of some constituent layers of an ICE design can degrade the ICE's target performance. In most cases, deviations of <0.1%, and even 0.01% or 0.0001%, from point by point design values of the optical characteristics (e.g., complex refractive indices), and/or physical characteristics (e.g., thicknesses) of the formed layers of the ICE can reduce the ICE's performance, in some cases to such an extent, that the ICE becomes operationally useless. Examples of fabrication errors include differences between values of complex refractive indices of layers of the ICE as conventionally fabricated—e.g., by reactive magnetron sputtering at room temperature—and as used in a down-hole optical analysis tool—at elevated temperature. In such cases, although complex refractive indices and thicknesses of the layers are found to be on target as fabrication of the ICE is completed at room temperature, the ICE materials' complex refractive indices change as a function of temperature, for some materials significantly, when the fabricated ICE is operated at an operational temperature much higher than the room temperature at which the ICE was fabricated. Such changes in the complex refractive indices of the ICE layers due to differences between fabrication and operational temperatures lead to temperature-dependent performance degradation for the conventionally fabricated ICE. Those familiar or currently practicing in the art will readily appreciate that the ultra-high accuracies required by ICE designs challenge the state of the art in thin film fabrication techniques.

### DESCRIPTION OF DRAWINGS

FIGS. 1A-1C show multiple configurations of an example of a system for analyzing wellbore fluids that uses a well logging tool including an ICE.

FIG. 2 is a flowchart showing an example of a process for designing an ICE.

FIGS. 3A-3C show multiple configurations of an example of a system for fabricating one or more ICEs in which temperature of the ICE(s) being fabricated is controlled.

FIGS. 4A-4I show aspects of ICE fabrication at temperatures lower than an annealing temperature of the ICE(s).

FIGS. 5A-5D show aspects of ICE fabrication at temperatures higher than the annealing temperature of the ICE(s).

FIG. 6 is a flowchart showing an example of an ICE fabrication during which temperature of ICEs being fabricated is controlled.

Like reference symbols in the various drawings indicate like elements.

### DETAILED DESCRIPTION

Technologies are described for controlling temperature of ICEs during ICE fabrication. For example, temperature of substrates of the ICEs is maintained at a target fabrication temperature by heating a substrate support—that supports the ICEs during fabrication—through electrical conductive heating elements that are part of the substrate support, inductive elements that are adjacent the substrate support, radiative elements (e.g., black body, laser, etc.) that are spaced apart from the substrate support, and the like. In some implementations, the target fabrication temperature at which layers of the ICEs are formed is the operational temperature. In these cases, performance of the fabricated ICEs will be above a minimum required performance at least for temperatures in the vicinity of the operational temperature. In some implementations, the target fabrication temperature at which the layers of the ICEs are formed exceeds an annealing temperature of the constituent materials of the ICE layers. The annealing temperature of a material is a temperature at which the material irreversibly transitions from a stressed state below the annealing temperature to an annealed (stress-relieved) state above the annealing temperature. The latter cases are used when it is required that performance of the fabricated ICEs exceeds the minimum required performance over a broad operational temperature range.

Prior to describing example implementations of the disclosed technologies for ICE fabrication, the following technologies are described below: in Section (1)—optical analysis tools based on ICE along with examples of their use in oil/gas exploration, and in Section (2)—techniques for designing an ICE.

### (1) ICE-Based Analysis of Wellbore Fluids

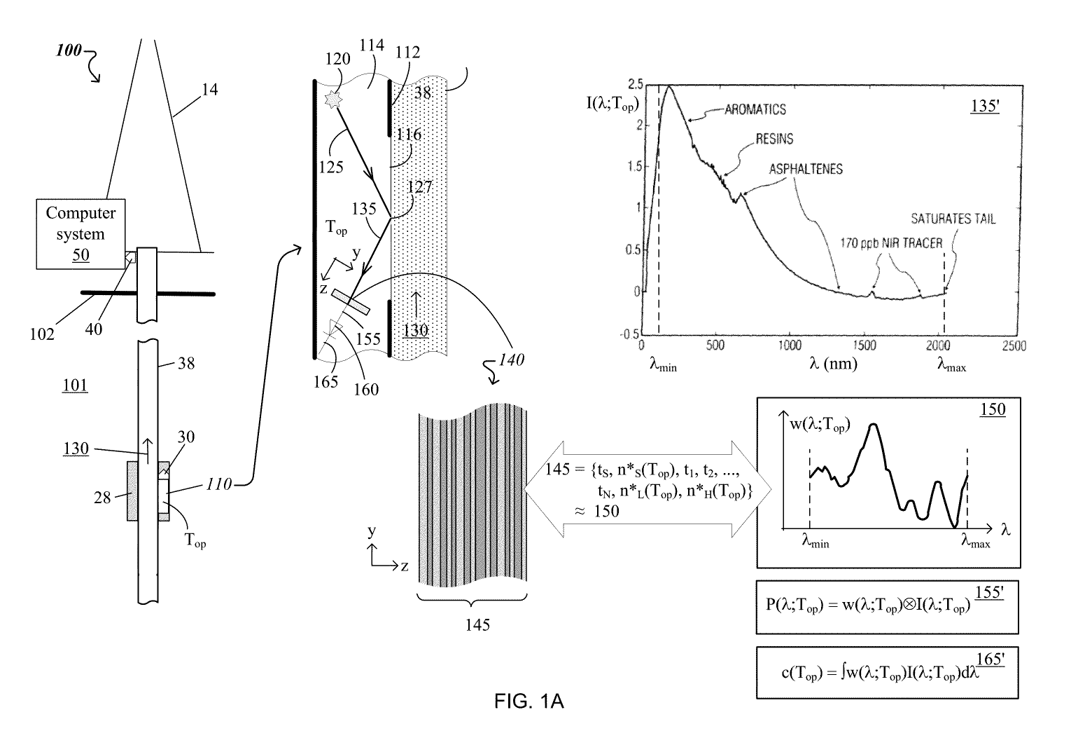

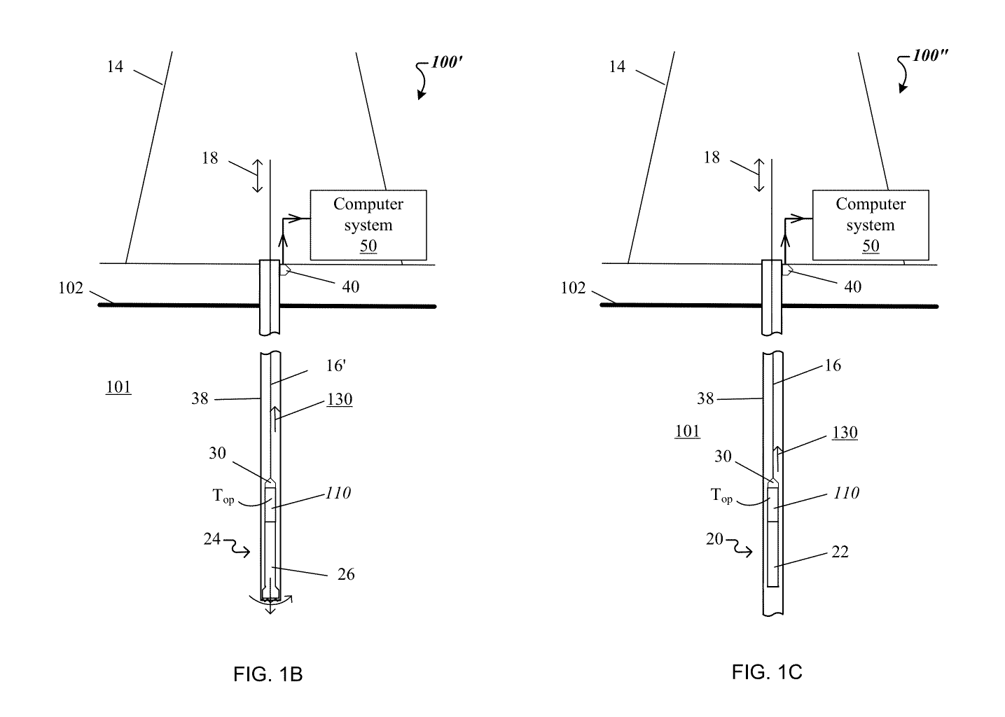

FIGS. 1A-1C show multiple configurations 100, 100′, 100″ of an example of a system for analyzing wellbore fluids 130, such that analyses are generated from measurements taken with a well logging tool 110 configured as an ICE-based optical analysis tool. The disclosed system also is referred to as a well logging system.

Each of the configurations 100, 100′, 100″ of the well logging system illustrated in FIGS. 1A-1C includes a rig 14 above the ground surface 102 and a wellbore 38 below the ground surface. The wellbore 38 extends from the ground surface into the earth 101 and generally passes through multiple geologic formations. In general, the wellbore 38 can contain wellbore fluids 130. The wellbore fluids 130 can be crude petroleum, mud, water or other substances and combinations thereof. Moreover, the wellbore fluids 130 may be at rest, or may flow toward the ground surface 102, for instance. Additionally, surface applications of the well logging tool 110 may include water monitoring and gas and crude transportation and processing.

FIG. 1A shows a configuration 100 of the well logging system which includes a permanent installation adjacent to the wellbore 38. In some implementations, the permanent installation is a set of casing collars that reinforce the wellbore 38. In this case, a casing collar 28 from among the set of casing collars supports the well logging tool 110 and a telemetry transmitter 30. A temperature of the wellbore fluids 130 increases as a function of distance (e.g., a depth) relative to the ground surface 102 based on a particular temperature gradient. E.g., the temperature at the ground surface 102 is substantially equal to the ambient temperature, T_{ambient}, has a value of approximately 150° C. adjacent the casing collar 28, and further increases at larger depths in the wellbore 38. In this manner, the well logging tool 110 operates at a constant operational temperature Tₒₚ adjacent the underground location of the casing collar 28 to determine and log properties of the wellbore fluids 130 at the operational temperature Tₒₚ.

FIG. 1B shows another configuration 100′ of the well logging system which includes a drilling tool 24 attached to a drill string 16′. The drilling tool 24 includes a drill bit 26, the ICE-based well logging tool 110 configured as a measurement while drilling (MWD) and/or logging while drilling (LWD) tool, and the telemetry transmitter 30. Drilling mud is provided through the drill string 16′ to be injected into the borehole 38 through ports of the drill bit 26. The injected drilling mud flows up the borehole 38 to be returned above the ground level 102, where the returned drilling mud can be resupplied to the drill string 16′ (not shown in FIG. 1B). In this case, the MWD/LWD-configured well logging tool 110 generates and logs information about the wellbore fluids 130 (e.g., drilling mud in this case) adjacent the working drill bit 26 at an operational temperature Tₒₚ that depends on drilling-related factors such as vertical speed and rotation speed of the drill bit 26, hardness of formation that currently being drilled, heat transfer properties of the formation and of the drilling mud, and the like. Here, the operational temperature Tₒₚ also depends on distance (e.g., depth) of the drilling tool 24 relative the ground level 102. For these reasons, the operational temperature Tₒₚ is significantly higher than the ambient temperature T_{ambient} and may be changing based on the foregoing environmental parameters adjacent the drill bit 26.

FIG. 1C shows yet another configuration 100″ of the well logging system which includes a tool string 20 attached to a cable 16 that can be lowered or raised in the wellbore 38 by draw works 18. The tool string 20 includes measurement and/or logging tools to generate and log information about the wellbore fluids 130 in the wellbore 38. In the configuration 100″ of the well logging system, this information is generated as a function of a distance (e.g., a depth) with respect to the ground surface 102. Moreover, the operational temperature Tₒₚ of the tool string 20 varies continuously as a function of wellbore depth, and thus the information about the wellbore fluids 130 in the wellbore 38 generated by the tool string 20 is temperature dependent. In the example illustrated in FIG. 1C, the tool string 20 includes the well logging tool 110, one or more additional well logging tool(s) 22, and the telemetry transmitter 30. Each of the well logging tools 110 and 22 measures one or more properties of the wellbore fluids 130. In some implementations, the well logging tool 110 determines values of the one or more properties in real time and reports those values instantaneously as they occur in the flowing stream of wellbore fluids 130, sequentially to or simultaneously with other measurement/logging tools 22 of the tool string 20.

In each of the above configurations 100, 100′ and 100″ of the well logging system, the values of the one or more properties measured by the well logging tool 110 are provided (e.g., as a detector signal 165) to the telemetry transmitter 30. The latter communicates the measured values to a telemetry receiver 40 located above the ground surface 102. The telemetry transmitter 30 and the telemetry receiver 40 can communicate through a wired or wireless telemetry channel. In some implementations of the system configurations 100′, 100″ illustrated in FIGS. 1B and 1C, e.g., in slickline or coiled tubing applications, measurement data generated by the well logging tool 110 can be written locally to memory of the well logging tool 110.

The measured values of the one or more properties of the wellbore fluids 130 received by the telemetry receiver 40 can be logged and analyzed by a computer system 50 associated with the rig 14. In this manner, the measurement values provided by the well logging tool 110 can be used to generate physical and chemical information about the wellbore fluids 130 in the wellbore 38 as a function of temperature, for instance.

Referring again to FIG. 1A, the well logging tool 110 includes a light source 120, an ICE 140 and an optical transducer 160. The well logging tool 110 has a frame 112 such that these components are arranged in an enclosure 114 thereof. A temperature inside the enclosure 114 is the operational temperature Tₒₚ. A cross-section of the well logging tool 110 in a plane perpendicular to the page can vary, depending on the space available. For example, the well logging tool's cross-section can be circular or rectangular, for instance. The well logging tool 110 directs light to the sample 130 through an optical interface 116, e.g., a window in the frame 112. The well logging tool 110 is configured to probe the sample 130 (e.g., the wellbore fluids stationary or flowing) in the wellbore 38 through the optical interface 116 and to determine an amount (e.g., a value) of a given characteristic (also referred to as a characteristic to be measured) of the probed sample 130 at the operational temperature Tₒₚ. The characteristic to be measured can be any one of multiple characteristics of the sample 130 including concentration of a given substance in the sample, a gas-oil-ratio (GOR), pH value, density, viscosity, etc.

The light source 120 outputs light with a source spectrum over a particular wavelength range, from a minimum wavelength λₘᵢₙ to a maximum wavelength λₘₐₓ. In some implementations, the source spectrum can have non-zero intensity over the entire or most of the wavelength range λₘₐₓ−λₘᵢₙ. In some implementations, the source spectrum extends through UV-vis (0.2-0.8 μm) and near-IR (0.8-2.5 μm) spectral ranges. Alternatively, or additionally, the source spectrum extends through near-IR and mid-IR (2.5-25 μm) spectral ranges. In some implementations, the source spectrum extends through near-IR, mid-IR and far-IR (25-100 μm) spectral ranges. In some implementations, the light source 120 is tunable and is configured in combination with time resolved signal detection and processing.

The light source 120 is arranged to direct a probe beam 125 of the source light towards the optical interface 116 where it illuminates the sample 130 at a location 127. The source light in the probe beam 125 interacts with the sample 130 and reflects off it as light modified by the sample 130. The light modified by the sample at Tₒₚ has a modified spectrum I(λ;Tₒₚ) 135′ over the particular wavelength range. In the reflective configuration of the well logging tool 110 illustrated in FIG. 1A (i.e., where the light to be analyzed reflects at the sample/window interface), the modified spectrum I(λ;Tₒₚ) 135′ is a reflection spectrum associated with the sample 130. In a transmission configuration of the well logging tool 110 (not shown in FIG. 1A), the probe beam is transmitted through the sample as sample modified light, such that the modified spectrum I(λ;Tₒₚ) 135′ is a transmission spectrum associated with the sample.

In general, the modified spectrum I(λ;Tₒₚ) 135′ encodes information about multiple characteristics associated with the sample 130, and more specifically the encoded information relates to current values of the multiple characteristics at the operational temperature Tₒₚ. In the example illustrated in FIG. 1A, the modified spectrum 135′ contains information about one or more characteristics of the wellbore fluids 130.

With continued reference to FIG. 1A, and the Cartesian coordinate system provided therein for reference, the ICE 140 is arranged to receive a beam 135 of the sample modified light, and is configured to process it and to output a beam 155 of processed light. The beam 135 of sample modified light is incident on a first surface of the ICE 140 along the z-axis, and the beam 155 of processed light is output along the z-axis after transmission through the ICE 140. Alternatively or additionally, the beam 155 (or an additional reflected beam) of processed light can be output after reflection off the first surface of the ICE 140. The ICE 140 is configured to process the sample modified light by weighting it in accordance with an optical spectrum w(λ;Tₒₚ) 150 associated with a characteristic to be measured at the operational temperature Tₒₚ.

The optical spectrum w(λ;Tₒₚ) 150 is determined offline by applying conventional processes to a set of calibration spectra I(λ;Tₒₚ) of the sample which correspond to respective known values at Tₒₚ of the characteristic to be measured. As illustrated by optical spectrum w(λ;Tₒₚ) 150, optical spectrums generally may include multiple local maxima (peaks) and minima (valleys) between λₘᵢₙ and λₘₐₓ. The peaks and valleys may have the same or different amplitudes. For instance, an optical spectrum w(λ;Tₒₚ) can be determined through regression analysis of N_{c} calibration spectra Iⱼ(λ;Tₒₚ) of a sample, where j=1, . . . , N_{c}, such that each of the calibration spectra Iⱼ(λ;Tₒₚ) corresponds to an associated known value at Tₒₚ of a given characteristic for the sample. A typical number N_{c} of calibration spectra Iⱼ(λ;Tₒₚ) used to determine the optical spectrum w(λ;Tₒₚ) 150 through such regression analysis can be N_{c}=10, 40 or 100, for instance. The regression analysis outputs, using the N_{c} calibration spectra Iⱼ(λ;Tₒₚ) as inputs, a spectral pattern that is unique to the given characteristic at Tₒₚ. The spectral pattern output by the regression analysis corresponds to the optical spectrum w(λ;Tₒₚ) 150. In this manner, when a value of the given characteristic for the sample is unknown at Tₒₚ, a modified spectrum Iᵤ(λ;Tₒₚ) of the sample is acquired at Tₒₚ and then the modified spectrum Iᵤ(λ;Tₒₚ) is weighted by the ICE 140 to determine a magnitude of the spectral pattern corresponding to the optical spectrum w(λ;Tₒₚ) 150 within the modified spectrum Iᵤ(λ;Tₒₚ). The determined magnitude is proportional to the unknown value at Tₒₚ of the given characteristic for the sample.

For example, the sample can be a mixture (e.g., the wellbore fluid 130 at Tₒₚ) containing substances X, Y and Z, and the characteristic to be measured for the mixture is concentration c_{X} of substance X in the mixture. In this case, N_{c} calibration spectra Iⱼ(λ;Tₒₚ) were acquired for N_{c} samples of the mixture having respectively known concentration values at Tₒₚ for each of the substances contained in the N_{c} samples. By applying regression analysis to the N_{c} calibration spectra Iⱼ(λ;Tₒₚ), a first spectral pattern that is unique to the concentration c_{X} of the X substance at Tₒₚ can be detected (recognized), such that the first spectral pattern corresponds to a first optical spectrum w_{cX}(λ;Tₒₚ) associated with a first ICE, for example. Similarly, second and third spectral patterns that are respectively unique to concentrations c_{Y} and c_{Z} of the Y and Z substances at Tₒₚ can also be detected, such that the second and third spectral patterns respectively correspond to second and third optical spectra w_{cY}(λ;Tₒₚ) and w_{c}(λ;Tₒₚ) respectively associated with second and third ICEs. In this manner, when a new sample of the mixture (e.g., the wellbore fluid 130 at Tₒₚ) has an unknown concentration c_{X} of the X substance, for instance, a modified spectrum Iᵤ(λ;Tₒₚ) of the new sample can be acquired at Tₒₚ by interacting the probe beam with the mixture, then the modified spectrum Iᵤ(λ;Tₒₚ) is weighted with the first ICE to determine a magnitude of the first spectral pattern within the modified spectrum Iᵤ(λ;Tₒₚ). The determined magnitude is proportional to the unknown value at Tₒₚ of the concentration c_{X} of the X substance for the new sample.

Referring again to FIG. 1A, the ICE 140 includes N layers of materials stacked on a substrate, such that complex refractive indices of adjacent layers are different from each other. The total number of stacked layers can be between 6 and 50, for instance. The substrate material can be BK7, diamond, Ge, ZnSe (or other transparent dielectric material), and can have a thickness in the range of 0.02-2 mm, for instance, to insure structural integrity of the ICE 140.

Throughout this specification, a complex index of refraction (or complex refractive index) n* of a material has a complex value, Re(n*)+iIm(n*). Re(n*) represents a real component of the complex index of refraction responsible for refractive properties of the material, and Im(n*) represents an imaginary component of the complex index of refraction (also known as extinction coefficient κ) responsible for absorptive properties of the material. In this specification, when it is said that a material has a high complex index of refraction n*_{H} and another material has a low complex index of refraction n*_{L}, the real component Re(n*_{H}) of the high complex index of refraction n*_{H} is larger than the real component Re(n*_{L}) of the low complex index of refraction n*_{L}, Re(n*_{H})>Re(n*_{L}). Materials of adjacent layers of the ICE are selected to have a high complex index of refraction n*_{H} (e.g., Si), and a low complex index of refraction n*_{L} (e.g., SiO₂). Here, Re(n*_{Si})≈2.4>Re(n*_{SiO2})≈1.5. For other material pairings, however, the difference between the high complex refractive index n*_{H} and low complex refractive index n*_{L} may be much smaller, e.g., Re(n*_{H})≈1.6>Re(n*_{L})≈1.5. The use of two materials for fabricating the N layers is chosen for illustrative purposes only. For example, a plurality of materials having different complex indices of refraction, respectively, can be used. Here, the materials used to construct the ICE are chosen to achieve a desired optical spectrum w(λ) 150.

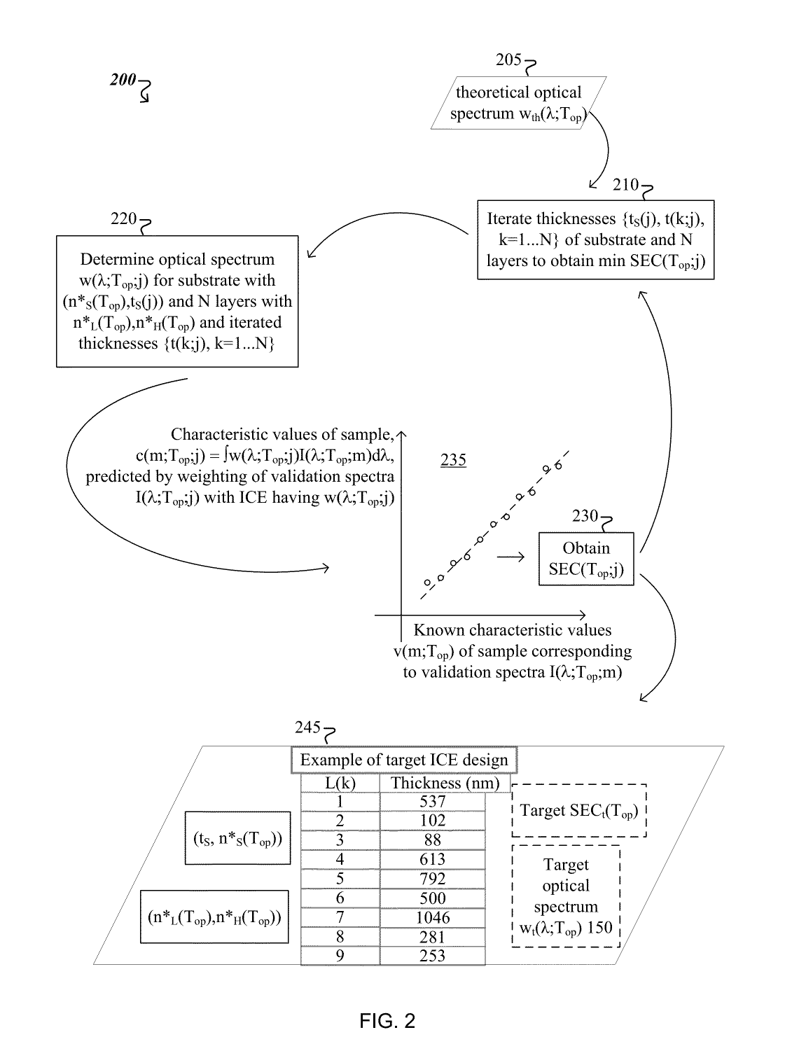

A set of design parameters 145—which includes the total number of stacked layers N, the complex refractive indices n*_{H}(Tₒₚ), n*_{L}(Tₒₚ) at Tₒₚ of adjacent stacked layers, and the thicknesses of the N stacked layers t(1), t(2), . . . , t(N−1), t(N)—of the ICE 140 can be chosen (as described below in connection with FIG. 2) to be spectrally equivalent, at Tₒₚ, to the optical spectrum w(λ;Tₒₚ) 150 associated with the characteristic to be measured. As such, an ICE design 145 is the set of thicknesses {t(i), i=1, . . . , N} of the N layers stacked on the substrate and their alternating complex refractive indices n*_{H}(Tₒₚ), n*_{L}(Tₒₚ) at Tₒₚ that corresponds to the optical spectrum w(λ;Tₒₚ) 150.

In view of the above, the beam 155 of processed light output by the ICE 140 has a processed spectrum P(λ;Tₒₚ)=w(λ;Tₒₚ) [symbol] I(λ;Tₒₚ) 155′ over the wavelength range λₘₐₓ−λₘᵢₙ at Tₒₚ, such that the processed spectrum 155′ represents the modified spectrum I(λ;Tₒₚ) 135′ weighted by the optical spectrum w(λ;Tₒₚ) 150 associated with the characteristic to be measured.

The beam 155 of processed light is directed from the ICE 140 to the optical transducer 160, which detects the processed light and outputs a detector signal 165. A value (e.g., a voltage) of the detector signal 165 is a result of an integration of the processed spectrum 155′ over the particular wavelength range and is proportional to the unknown value c(Tₒₚ) 165′ at Tₒₚ of the characteristic to be measured for the sample 130.

In some implementations, the well logging tool 110 can include a second ICE (not shown in FIG. 1A) associated with a second ICE design that includes a second set of thicknesses {t′(i), i=1, . . . , N′} of a second total number of layers N′ layers with alternating complex refractive indices (n*′_{H}(Tₒₚ),n*′_{L}(Tₒₚ)) at Tₒₚ stacked on a second substrate that correspond to a second optical spectrum w′(λ;Tₒₚ). Here, the second optical spectrum w′(λ;Tₒₚ) is associated with a second characteristic of the sample 130 at Tₒₚ, and a second processed spectrum represents the modified spectrum I(λ;Tₒₚ) 135′ weighted by the second optical spectrum w′(λ;Tₒₚ), such that a second value of a second detector signal is proportional to a value at Tₒₚ of the second characteristic for the sample 130.

In some implementations, the determined value 165′ of the characteristic to be measured can be logged along with the operational temperature Tₒₚ, a measurement time, geo-location, and other metadata, for instance. In some implementations, the detector signal 165, which is proportional to a characteristic to be measured by the well logging tool 110, can be used as a feedback signal to adjust the characteristic of the sample, to modify the sample or environmental conditions associated with the sample, as desired.

Characteristics of the wellbore fluids 130 that can be related to the modified spectrum 135′ through the optical spectra associated with the ICE 140 and other ICEs (not shown in FIG. 1A) are concentrations of one of asphaltene, saturates, resins, aromatics; solid particulate content; hydrocarbon composition and content; gas composition C1-C6 and content: CO₂, H₂S and correlated PVT properties including GOR, bubble point, density; a petroleum formation factor; viscosity; a gas component of a gas phase of the petroleum; total stream percentage of water, gas, oil, solid articles, solid types; oil finger printing; reservoir continuity; oil type; and water elements including ion composition and content, anions, cations, salinity, organics, pH, mixing ratios, tracer components, contamination, or other hydrocarbon, gas, solids or water characteristic.

### (2) Aspects of ICE Design

Aspects of a process for designing an ICE associated with a characteristic (e.g., one of the characteristics enumerated above) to be measured at an operational temperature Tₒₚ are described below. Here, an input of the ICE design process is a theoretical optical spectrum wₜₕ(λ;Tₒₚ) associated with the characteristic. An output of the ICE design process is an ICE design that includes specification of (1) a substrate and a number N of layers to be formed on the substrate, each layer having a different complex refractive index from its adjacent layers; and (2) complex refractive indices and thicknesses of the substrate and layers that correspond to a target optical spectrum wₜ(λ;Tₒₚ). The target optical spectrum wₜ(λ;Tₒₚ) is different from the theoretical optical spectrum wₜₕ(λ;Tₒₚ) associated with the characteristic at Tₒₚ, such that the difference between the target and theoretical optical spectra cause degradation of a target performance relative to a theoretical performance of the ICE within a target error tolerance. In this example, the target performance represents a finite accuracy with which an ICE having the target optical spectrum wₜ(λ;Tₒₚ) is expected to predict known values at Tₒₚ of the characteristic corresponding to a set of validation spectra of a sample with a finite (non-zero) error. Here, the predicted values of the characteristic are obtained through integration of the validation spectra of the sample respectively weighted by the ICE with the target optical spectrum wₜ(λ;Tₒₚ). The theoretical performance represents the maximum accuracy with which the ICE—if it had the theoretical optical spectrum wₜₕ(λ;Tₒₚ)—would predict the known values at Tₒₚ of the characteristic corresponding to the set of validation spectra of the sample. Here, the theoretically predicted values of the characteristic would be obtained through integration of the validation spectra of the sample respectively weighted by the ICE, should the ICE have the theoretical optical spectrum wₜₕ(λ;Tₒₚ).

FIG. 2 is a flowchart of an example of a process 200 for generating an ICE design. One of the inputs to the process 200 is a theoretical optical spectrum wₜₕ(λ;Tₒₚ) 205. For instance, to design an ICE for measuring concentration of a substance X in a mixture at Tₒₚ, a theoretical optical spectrum wₜₕ(λ;Tₒₚ), associated with the concentration of the substance X in the mixture, is accessed, e.g., in a data repository. As described above in this specification, the accessed theoretical optical spectrum wₜₕ(λ;Tₒₚ) corresponds to a spectral pattern detected offline, using a number N_{c} of calibration spectra of the mixture, each of the N_{c} calibration spectra corresponding to a known concentration at Tₒₚ of the substance X in the mixture. An additional input to the process 200 is a specification of materials for the ICE layers. Materials having different complex refractive indices at Tₒₚ, respectively, are specified such that adjacent ICE layers are formed from materials with different complex refractive indices. For example, a first material (e.g., Si) having a high complex refractive index n*_{H} and a second material (SiOₓ) having a low complex refractive index n*_{L} are specified to alternately form ICE layers. As another example, a layer can be made from high index material (e.g., Si), followed by a layer made from low index material (e.g., SiOₓ), followed by a layer made from a different high index material (e.g., Ge), followed by a layer made from a different low index material (MgF₂), etc. The iterative design process 200 is performed in the following manner.

At 210 during the j^{th} iteration of the design process 200, thicknesses {t_{S}(j), t(1;j), t(2;j), . . . , t(N−1;j), t(N;j)} of the substrate and a number N of layers of the ICE are iterated.

At 220, a j^{th} optical spectrum w(λ;Tₒₚ;j) of the ICE is determined corresponding to complex refractive indices (n*_{L}(Tₒₚ),n*_{H}(Tₒₚ)) at Tₒₚ and previously iterated thicknesses {t_{S}(j), t(1;j), t(2;j), . . . , t(N−1;j), t(N;j)} of the substrate and the N layer, each having a different complex refractive index from is adjacent layers. The iterated thicknesses of the substrate and the N layers are used to determine the corresponding j^{th} optical spectrum w(λ;Tₒₚ;j) of the ICE in accordance with conventional techniques for determining spectra of thin film interference filters.

At 230, performance of the ICE, which has the j^{th} optical spectrum w(λ;Tₒₚ;j) determined at 220, is obtained. To do so, a set of validation spectra taken at Tₒₚ of a sample is accessed, e.g., in a data repository. Respective values at Tₒₚ of a characteristic of the sample are known for the validation spectra. For instance, each of Nᵥ validation spectra I(λ;Tₒₚ;m) corresponds to a value v(m;Tₒₚ) at Tₒₚ of the characteristic of the sample, where m=1, . . . , Nᵥ. In the example illustrated in FIG. 2, Nᵥ=11 validation spectra, respectively corresponding to 11 known values of the characteristic to be measured for the sample, are being used.

Graph 235 shows (in open circles) values c(m;Tₒₚ;1) at Tₒₚ of the characteristic of the sample predicted by integration of the validation spectra I(λ;Tₒₚ;m) processed by the ICE, which has the j^{th} optical spectrum w(λ;Tₒₚ;j), plotted against the known values v(m;Tₒₚ) at Tₒₚ of the characteristic of the sample corresponding to the validation spectra I(λ;Tₒₚ;m). The predicted values c(m;Tₒₚ;1) of the characteristic are found by substituting, in formula 165′ of FIG. 1A, (1) the spectrum I(λ;Tₒₚ) 135′ of sample modified light with the respective validation spectra I(λ;Tₒₚ;m) and (2) the target spectrum wₜ(λ;Tₒₚ) 150 with the j^{th} optical spectrum w(λ;Tₒₚ;1). In this example, performance of the ICE at Tₒₚ, which has the j^{th} optical spectrum w(λ;Tₒₚ;j), is quantified in terms of a weighted measure of distances from each of the open circles in graph 235 to the dashed-line bisector between the x and y axes. This weighted measure is referred to as the standard calibration error of the ICE at Tₒₚ, SEC(Tₒₚ). For instance, an ICE having the theoretical spectrum wₜₕ(λ;Tₒₚ) has a theoretical SECₜₕ(Tₒₚ) that represents a lower bound for the SEC(Tₒₚ;j) of the ICE having the j^{th} spectrum w(λ;Tₒₚ;j) determined at 220 during the j^{th} iteration of the design process 200: SEC(Tₒₚ;j)>SECₜₕ(Tₒₚ).

In this specification, the SEC is chosen as a metric for evaluating ICE performance for the sake of simplicity. Note that there are other figures of merit that may be used to evaluate performance of ICE, as is known in the art. For example, sensitivity—which is defined as the slope of characteristic change as a function of signal strength—can also be used to evaluate ICE performance. As another example, standard error of prediction (SEP)—which is defined in a similar manner to the SEC except it uses a different set of validation spectra—can be used to evaluate ICE performance. Any of the figure(s) of merit known in the art is/are evaluated in the same general way by comparing theoretical performance with that actually achieved. Which figure(s) of merit or combinations are used to evaluate ICE performance is determined by the specific ICE design.

The iterative design process 200 continues by iterating, at 210, the thicknesses of the substrate and the N layers. The iterating is performed such that a (j+1)^{th} optical spectrum w(λ;Tₒₚ;j+1)—determined at 220 from the newly iterated thicknesses—causes, at 230, improvement in performance of the ICE, to obtain SEC(Tₒₚ;j+1)<SEC(Tₒₚ;j). In some implementations, the iterative design process 200 is stopped when the ICE's performance at Tₒₚ reaches a local maximum, or equivalently, the SEC of the ICE reaches a local minimum. For example, the iterative process 200 can be stopped at the (j+1)^{th} iteration when the current SEC(Tₒₚ;j+1) is larger than the last SEC(Tₒₚ;j), SEC(Tₒₚ;j+1)>SEC(Tₒₚ;j). In some implementations, the iterative design process 200 is stopped when, for a given number of iterations, the ICE's performance exceeds a specified threshold performance for a given number of iterations. For example, the iterative design process 200 can be stopped at the j^{th} iteration when three consecutive SEC values decrease monotonously and are less than a specified threshold value: SEC₀>SEC(Tₒₚ;j−2)>SEC(Tₒₚ;j−1)>SEC(Tₒₚ;j).

In either of these cases, an output of the iterative process 200 represents a target ICE design 245 to be used for fabricating an ICE 140, like the one described in FIG. 1A, for instance. The ICE design 245 includes specification of (1) a substrate and N layers, each having a different complex refractive index from its adjacent layers, and (2) complex refractive indices n*_{S}(Tₒₚ), n*_{H}(Tₒₚ), n*_{L}(Tₒₚ) at Tₒₚ and thicknesses {t_{S}(j), t(1;j), t(2;j), . . . , t(N−1;j), t(N;j)} of the substrate and N layers corresponding to the j^{th} iteration of the process 200. Additional components of the ICE design are the optical spectrum w(λ;Tₒₚ;j) and the SEC(Tₒₚ;j)—both determined during the j^{th} iteration based on the thicknesses {t_{S}(j), t(1;j), t(2;j), . . . , t(N−1;j), t(N;j)}. As the ICE design 245 is used as input for fabrication processes described herein, the iteration index j—at which the iterative process 200 terminates—is dropped from the notations used for the components of the ICE design.

In this manner, the thicknesses of the substrate and the N layers associated with the ICE design 245 are denoted {t_{S}, t(1), t(2), . . . , t(N−1), t(N)} and are referred to as the target thicknesses; the complex refractive indices (n*_{L}(Tₒₚ),n*_{H}(Tₒₚ)) at Tₒₚ are referred to as target complex refractive indices. The optical spectrum associated with the ICE design 245 and corresponding to the target thicknesses is referred to as the target optical spectrum wₜ(λ;Tₒₚ) 150. The SEC associated with the ICE design 245—obtained in accordance with the target optical spectrum wₜ(λ;Tₒₚ) 150 corresponding to the target thicknesses—is referred to as the target SECₜ(Tₒₚ). In the example illustrated in FIG. 2, the ICE design 245 has a total of N=9 alternating Si and SiO₂ layers. The layers' thicknesses (in nm) are shown in the table. An ICE fabricated based on the example of ICE design 245 illustrated in FIG. 2 is used to predict value(s) of concentration of substance X in wellbore fluids 130 at an operational temperature Tₒₚ=150° C., for instance.

### (3) Technologies for Controlling Temperature of ICEs During Fabrication

As described above in connection with FIG. 2, an ICE design for fabricating ICEs to be operated at an operational temperature Tₒₚ (e.g., in a down-hole application) specifies a substrate and a number of material layers, each having a different complex refractive index from its adjacent layers. An ICE fabricated in accordance with such an ICE design has, when operated at Tₒₚ, (i) a target optical spectrum wₜ(λ;Tₒₚ) and (ii) a target performance SECₜ(Tₒₚ), both of which corresponding to the temperature-dependent complex refractive indices and target thicknesses of the substrate and the layers specified by the ICE design. Performance of the ICEs fabricated in accordance with an ICE design can be very sensitive to actual values of the complex refractive indices and thicknesses obtained during deposition, such that for some layers of the ICE design, a small error, e.g., 0.1% or 0.001%, in the optical or physical characteristics of a deposited layer can result in a reduction in the performance of an ICE associated with the ICE design below an acceptable threshold. For many reasons, the actual values of the complex refractive indices of materials to be deposited and/or the rate(s) of the deposition can drift when materials used for deposition (Si, SiO₂) are differently contaminated, or react differently due to different chamber conditions (e.g., pressure or temperature). As such, a temperature T_{fab} at which the ICEs are fabricated and the temperature(s) at which the ICEs are operated over (e.g., at Tₒₚ in a down-hole application) are correlated, and in some instances matched. As a practical matter, the temperature dependence of the complex refractive indices can be hard to predict. Hence, fabrication of ICEs to operate at high operational temperature Tₒₚ, or over a wide range of operational temperatures, is all the more challenging.

Conventionally, ICEs have been fabricated by reactive magnetron sputtering at ambient (e.g., room) temperature. ICEs fabricated using a particular ICE design—chosen based on a particular set of performance criteria (e.g., SEC, standard error in prediction (SEP), sensitivity, SNR, and/or theoretical temperature performance)—are subjected to ex-situ post-fabrication measurements to measure the ICEs' optical spectra wₜ(λ;T). Results of these ex-situ measurements are used to determine optical properties of the individual layer materials at various temperatures, e.g., n*_{H}(T), dn*_{H}/dT, and n*_{L}(T), dn*_{L}/dT. Such measurements generate information on how the ICEs will ultimately perform at the operational temperature(s) by extrapolation. Additionally, ICEs fabricated conventionally at ambient temperature to be used at elevated temperatures or over a broad temperature range, are annealed ex-situ (e.g., by placing the completed ICEs in a high temperature state for a period of time) to minimize ICE performance drift at elevated operational temperature(s) Tₒₚ. Such annealing—which may require additional measurements to determine changes in optical spectrum wₜ(λ;T) caused by the annealing process—further complicates conventional ICE fabrication.

The disclosed technologies relate to heating the ICEs' substrate during fabrication to eliminate (or move in-situ) parts of the ex-situ post-fabrication processing and analysis. Heating of the ICEs' substrate can be accomplished in-situ by conduction or radiation. Conduction heating techniques typically include adding conductive heating elements onto a substrate holder, usually a drum, plate or platen. Intensity of current through the conductive heating elements is adjusted to achieve a desired temperature of the ICEs' substrate. Radiative heating techniques include using an infrared (IR) emitter (e.g., a blackbody radiation emitter or an IR laser) that is spaced apart from the substrate holder or an inductive emitter that is adjacent the substrate holder. Both of the latter types of emitters are focused on one or more portions of the substrate holder to achieve a desired temperature of the ICEs' substrate.

The disclosed technologies can be used to fabricate ICEs to have a target optical spectrum and a corresponding ICE performance at an operational temperature Tₒₚ. As the optical properties of the materials used in fabricating ICEs are dependent on temperature, the ICEs' substrate temperature during deposition and the materials' temperature as they are being deposited are controlled to obtain complex refractive indices of the ICE layers with target values n*_{H}(Tₒₚ) n*_{L}(Tₒₚ) at the operational temperature Tₒₚ. These results lead to a desired ICE performance at the operational temperature Tₒₚ. For example, the ICEs' substrate temperature is raised to the expected operational temperature (e.g., downhole Tₒₚ=150° C.). Here, the ICE materials' optical properties can be monitored and controlled as the materials are deposited at the expected operational conditions. As another example, the ICEs' substrate temperature is used during deposition of the ICE layers as an extremely accurate and fine tunable control to obtain the complex refractive indices having target values n*_{H}(Tₒₚ) n*_{L}(Tₒₚ) at the operational temperature Tₒₚ. Here, changing the ICEs' substrate temperature during material deposition results in controlled values n*_{H}(T) or n*_{L}(T) of the complex refractive indices of a layer currently being deposited or of layers remaining to be deposited.

In this manner, the disclosed technologies enable ICEs to be designed and fabricated for use over a target operational temperature range more accurately and rapidly than conventional ICE design and fabrication. Details of one or more of the foregoing embodiments are described below.

### (3.1) System for ICE Fabrication that Allows for In-Situ Controlling Temperature of ICEs

Once a target ICE design is established to specify values of complex refractive indices n*_{H}(Tₒₚ), n*_{L}(Tₒₚ) corresponding to an operational temperature Tₒₚ at which ICEs are to be operated, the target ICE design can be provided to an ICE fabrication system in which one or more ICEs are fabricated based on the target ICE design. Technologies for controlling temperature of ICEs during fabrication are disclosed below to ensure accurate performance of the fabricated ICEs at the operational temperature Tₒₚ. A fabrication system for implementing these technologies is described first.

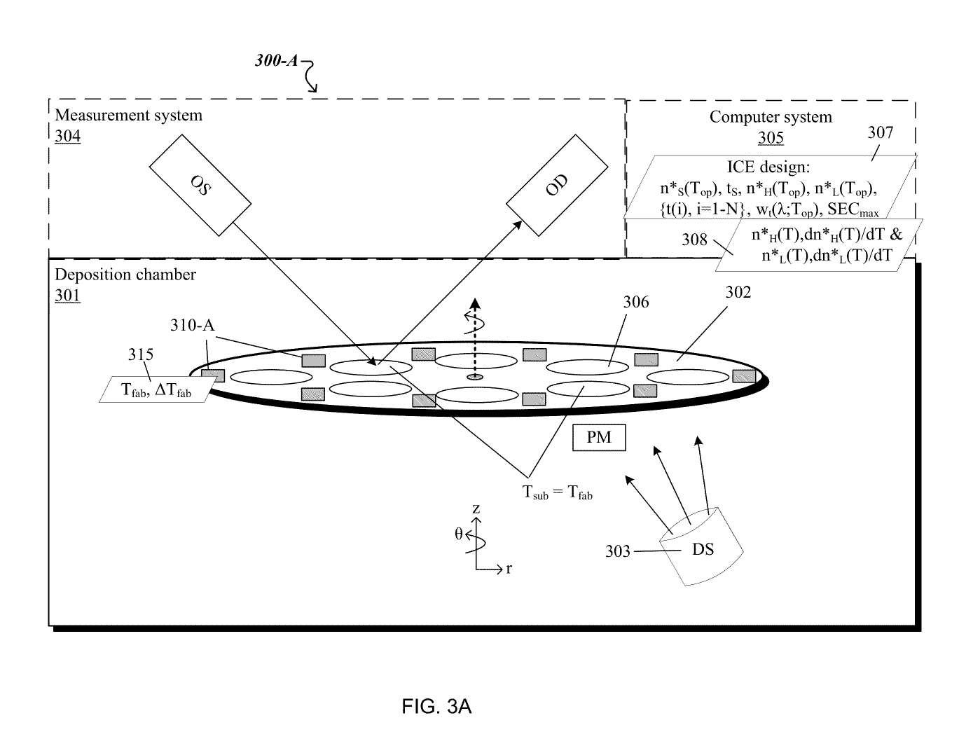

FIGS. 3A-3C shows different configurations of an example of an ICE fabrication system 300. The ICE fabrication system 300 includes a deposition chamber 301 to fabricate one or more ICEs 306, a measurement system 304 to measure characteristics of formed layers of the ICEs while the ICEs are being fabricated, and a computer system 305 to control the fabrication of the one or more ICEs 306 based at least in part on results of the measurements.

The deposition chamber 301 includes one or more deposition sources 303 to provide materials with a low complex index of refraction n*_{L} and a high complex index of refraction n*_{H} used to form layers of the ICEs 306. Substrates on which layers of the ICEs 306 will be deposited are placed on a substrate support 302, such that the ICEs 306 are within the field of view of the deposition source(s) 303. The substrates have a thickness t_{S} and a complex refractive index n*_{S}(Tₒₚ) specified by the ICE design 307. Various physical vapor deposition (PVD) techniques can be used to form a stack of layers of each of the ICEs 306 in accordance with a target ICE design 307 (e.g., ICE design 145 or 245, for instance.) Here, the ICE design 307 includes specification of a complex index of refraction n_{S}(Tₒₚ) at an operational temperature Tₒₚ and thickness t_{S} of a substrate; complex indices of refraction n*_{H}(Tₒₚ), n*_{L}(Tₒₚ) at Tₒₚ and target thicknesses {t(i), i=1−N} of N layers; and a corresponding target optical spectrum wₜ(λ;Tₒₚ), where λ is within an operational wavelength range [λₘᵢₙ, λₘₐₓ] of the ICEs.

In accordance with PVD techniques, the layers of the ICE are formed by condensation of a vaporized form of material(s) of the source(s) 305, while maintaining vacuum in the deposition chamber 301. One such example of PVD technique is electron beam (E-beam) deposition, in which a beam of high energy electrons is electromagnetically focused onto material(s) of the deposition source(s) 303, e.g., either Si, or SiO₂, to evaporate atomic species. In some cases, E-beam deposition is assisted by ions, provided by ion-sources (not shown in FIGS. 3A-3C), to clean or etch the ICE substrate(s); and/or to increase the energies of the evaporated material(s), such that they are deposited onto the substrates more densely, for instance. Other examples of PVD techniques that can be used to form the stack of layers of each of the ICEs 306 are cathodic arc deposition, in which an electric arc discharged at the material(s) of the deposition source(s) 303 blasts away some into ionized vapor to be deposited onto the ICEs 306 being formed; evaporative deposition, in which material(s) included in the deposition source(s) 303 is(are) heated to a high vapor pressure by electrically resistive heating; pulsed laser deposition, in which a laser ablates material(s) from the deposition source(s) 303 into a vapor; or sputter deposition, in which a glow plasma discharge (usually localized around the deposition source(s) 303 by a magnet—not shown in FIGS. 3A-3C) bombards the material(s) of the source(s) 303 sputtering some away as a vapor for subsequent deposition.

A relative orientation of and separation between the deposition source(s) 303 and the substrate support 302 are configured to provide desired deposition rate(s) and spatial uniformity across the ICEs 306 disposed on the substrate support 302. As a spatial distribution of a deposition plume provided by the deposition source(s) 303 is non-uniform along at least a first direction, current instances of ICEs 306 are periodically moved by the substrate support 302 relative to the deposition source 303 along the first direction (e.g., rotated along an azimuthal direction “θ” relative to an axis that passes through the deposition source(s) 303) to obtain reproducibly uniform layer deposition of the ICEs 306 within a batch.

A heating source 310 provides heat to the current instances of the ICEs 306 distributed on the substrate support 302 to maintain their temperature within a target fabrication temperature range ΔT_{fab} around a target fabrication temperature T_{fab}. A width of the target fabrication temperature range ΔT_{fab} is a fraction, e.g., 5%, 10%, 20%, or 30% of the target fabrication temperature T_{fab}. For instance, when the target fabrication temperature T_{fab}=150° C., the temperature range ΔT_{fab} can be [146.25° C., 153.75° C.], [142.5° C., 157.5° C.], [135° C., 165° C.] or [127.5° C., 172.5° C.]. A process parameter 315 that includes the target fabrication temperature T_{fab} and the target fabrication temperature range ΔT_{fab} is accessed by the computer system 305 and used to control the temperature of current instances of ICEs 306 during fabrication of ICEs associated with the ICE design 307.

In a configuration 310-A of the heating source associated with a configuration 300-A of the ICE fabrication system, the heating source includes electrical heating elements distributed throughout the substrate support 302 to maintain the target fabrication temperature T_{fab} of the current instances of ICEs 306 uniformly across the substrate support 302. An intensity of current carried through the electrical conductive heating elements is adjusted to obtain the target fabrication temperature T_{fab} for the current instances of ICEs 306.

In another configuration 310-B of the heating source associated with a configuration 300-B of the ICE fabrication system, the heating source includes an IR or blackbody radiation emitter placed apart from the substrate support 302 and focused on, at least, a portion of the substrate support 302. Here, the IR emitter can be an IR laser, for instance. A radiation flux (intensity per unit area) provided by the IR or blackbody radiation emitter onto the substrate support 302 is adjusted in conjunction with a period of rotation of the substrate support 302 to maintain the current instances of ICEs 306 across the substrate support 302 at the target fabrication temperature T_{fab}.

In yet another configuration 310-C of the heating source associated with a configuration 300-C of the ICE fabrication system, the heating source includes an inductive emitter disposed adjacent the substrate support 302 such that electromagnetic radiation provided by the inductive emitter is focused on, at least, a portion of the substrate support 302. The inductive emitter can be configured as one or more solenoids in a bipolar configuration, quadrupolar configuration, etc. A time-varying electromagnetic flux provided by the inductive emitter onto the substrate support 302 is adjusted in conjunction with the period of rotation of the substrate support 302 to maintain the current instances of ICEs 306 across the substrate support 302 at the target fabrication temperature T_{fab}.

The target fabrication temperature T_{fab} at which the current instances of the ICE 306 are heated during deposition is specified in the process parameter 315 such that complex refractive indices of layers of the fabricated ICE have target values n*_{H}(Tₒₚ), n*_{L}(Tₒₚ)—at the operational temperature Tₒₚ, or more generally, over an operational temperature range ΔTₒₚ, at or over which the fabricated ICEs will be operated—in accordance with the ICE design 307. The target fabrication temperature T_{fab} is correlated with the operational temperature Tₒₚ based on materials information 308 accessed by the computer system 305. The materials information 308 includes a predetermined temperature dependence n*_{H}(T) and n*_{L}(T) of the complex refractive indices of layers associated with the ICE design and their respective rate of change as a function of temperature dn*_{H}(T)/dT and dn*_{L}(T)/dT, over a temperature interval [Tₘᵢₙ, Tₘₐₓ]. Additionally, the materials information 308 includes a predetermined temperature dependence n*_{S}(T) of the complex refractive index of the substrate specified by the ICE design and its respective rate of change as a function of temperature dn*_{S}(T)/dT, over the temperature interval [Tₘᵢₙ, Tₘₐₓ]. Here, a temperature dependence of a complex refractive index n*(T) includes respective temperature dependencies for a real component of the complex refractive index n(T)=Re(n*(T)) and an imaginary component of the complex refractive index κ(T)=Im(n*(T)). Similarly, a rate of change of a complex refractive index dn*(T)/dT includes respective rates of change for a real component of the complex refractive index do/dT=d(Re(n*(T)))/dT and an imaginary component of the complex refractive index dκ/dT=d(Im(n*(T)))/dT with temperature. In some cases, Tₘᵢₙ is the temperature at the ground level 102 of the borehole 38 and Tₘₐₓ is 300° C. In other cases, Tₘᵢₙ=−40° C. and Tₘₐₓ is 400° C. The temperature ranges [Tₘᵢₙ, Tₘₐₓ] noted above can correspond to respective operational temperature ranges ΔTₒₚ associated with different applications of respective ICE designs. The foregoing materials information 308 can be used by the computer system 305 to control the heating source 310 for maintaining the temperature of the current instances of the ICEs 306 within a target fabrication temperature range ΔT_{fab} of a T_{fab} that is correlated with the Tₒₚ, as described in detail below.

For instance, the target fabrication temperature T_{fab} and range ΔT_{fab} depend on whether the ICEs 306 are fabricated to be used in an annealed state or an un-annealed state. As discussed above, an ICE is irreversibly annealed when heated at least through an upper bound of an annealing temperature range associated with the ICE design 307. For example, a finite (non-zero) annealing temperature range associated with the ICE design 307 is bound by an annealing temperature T_{AL} of a layer material with low complex refractive index n*_{L}(T) and an annealing temperature T_{AH} of an adjacent layer material with high complex refractive index n*_{H}(T). Here, a constituent material of the ICE with low/high complex refractive index n*_{L}(T)/n*_{H}(T) irreversibly transitions from a stressed state to an annealed (stress-relieved) state when heated through the annealing temperature T_{AL}/T_{AH}. As another example, the foregoing annealing temperature range collapses to a single annealing temperature T_{A} associated with the ICE design 307 if the stress is relieved—not in the bulk of the individual materials of the adjacent layers of the ICE, but—at the interface between the adjacent layers having complex refractive indices n*_{L}(T) and n*_{H}(T). Here the ICE irreversibly transitions from an interface-stressed state to an interface-annealed (stress-relieved) state when heated through the annealing temperature T_{A}.

### Example 1

In some implementations, ICEs are fabricated to be used in their un-annealed state at an operational temperature Tₒₚ over a narrow operational temperature range ΔTₒₚ, e.g., less than 30%, relative to its center value Tₒₚ. Un-annealed ICEs are exposed, both during and after fabrication, to temperatures that do not exceed the lower bound of the annealing temperature range.

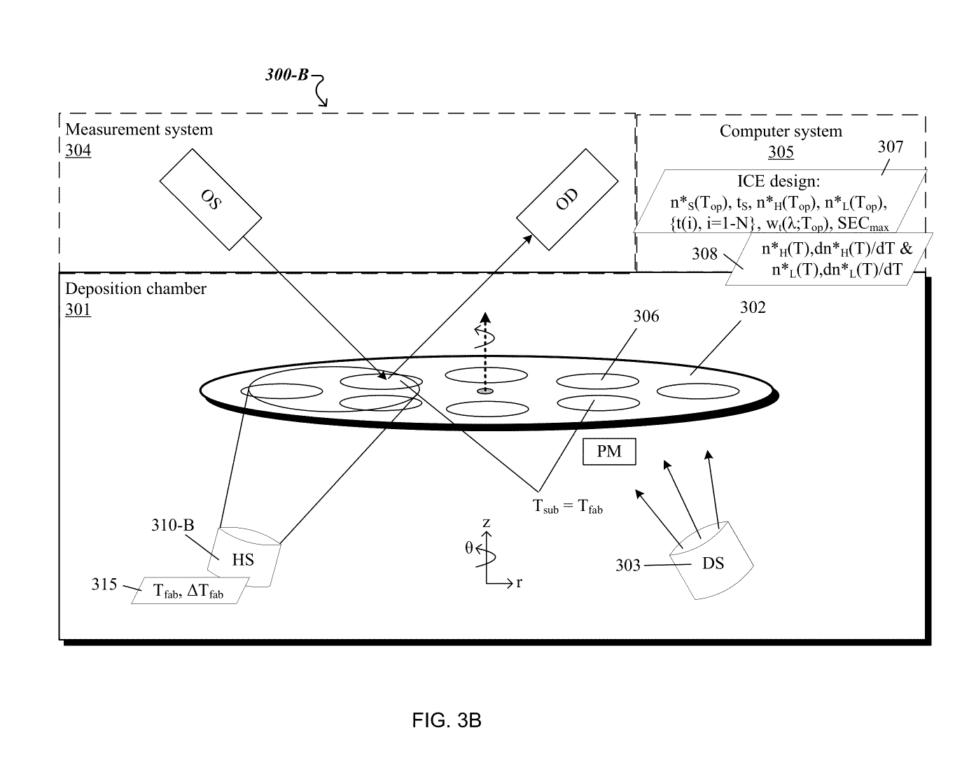

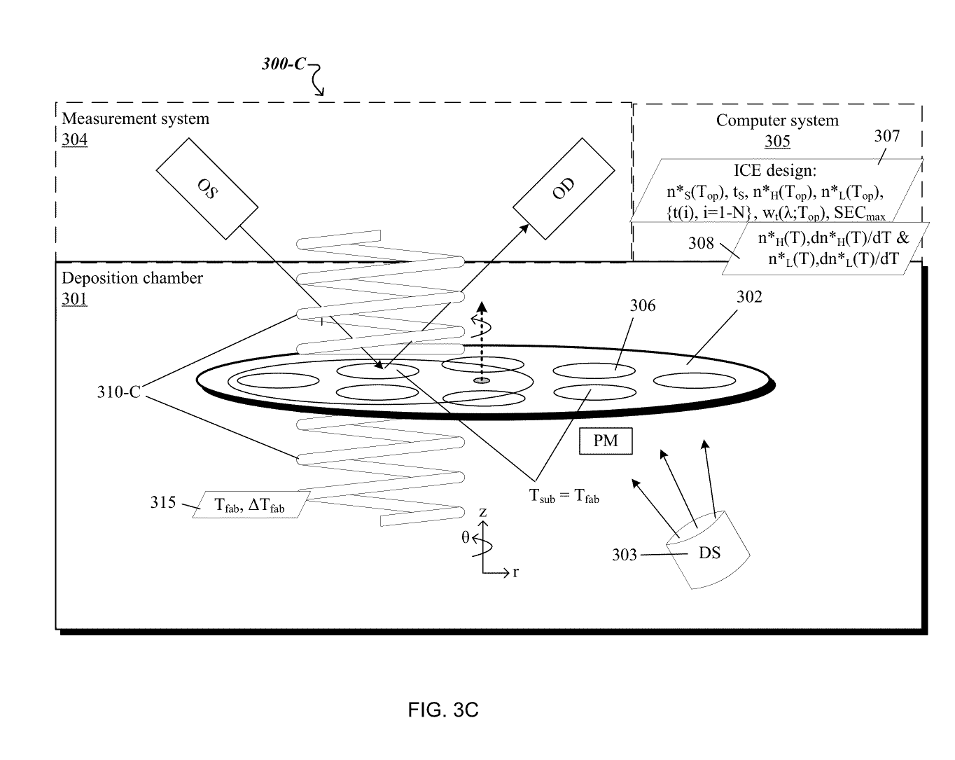

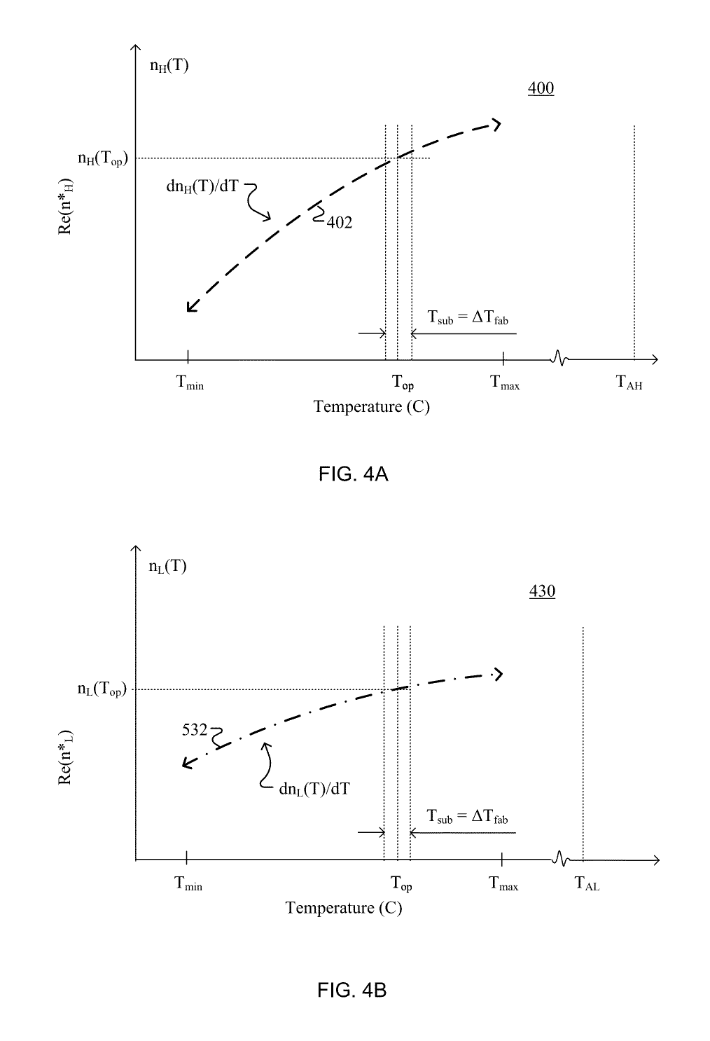

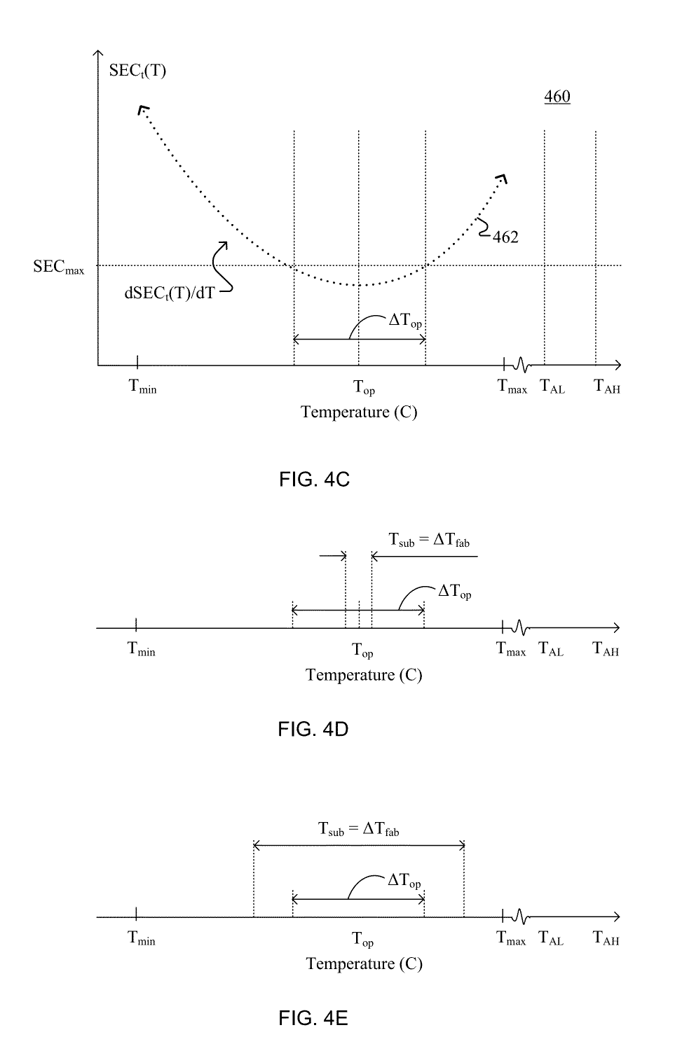

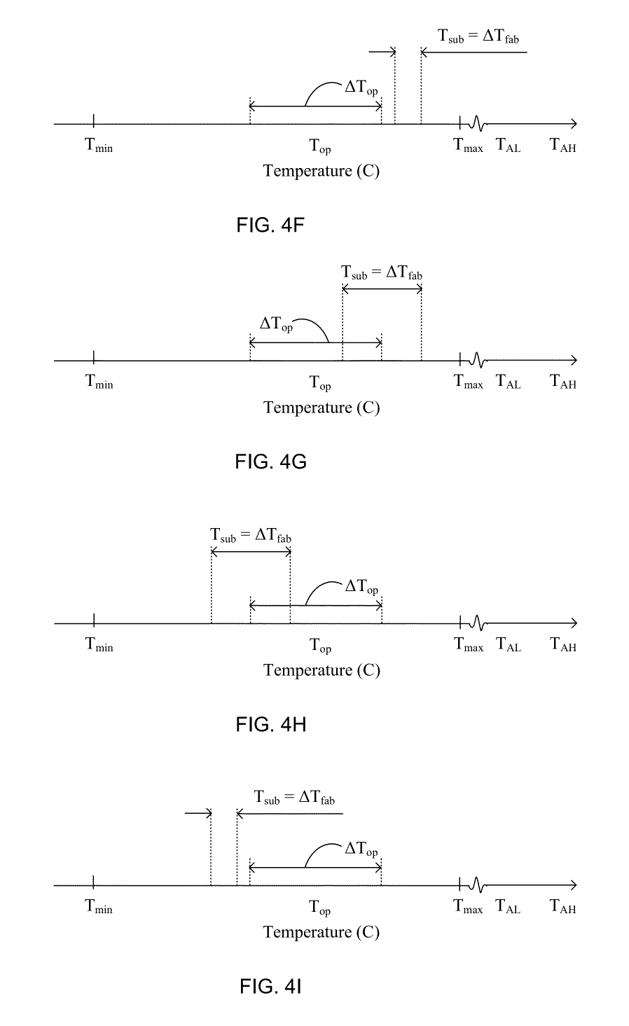

FIG. 4A shows a graph 400 in which a temperature dependence n_{H}(T) of real part of the high complex refractive index of a first material—from which some of the layers of the ICEs are formed—is represented as curve 402 for temperatures much lower than the annealing temperature T_{AH} of the first material, Tₘₐₓ<<T_{AH}. The arrows at both ends of curve 402 signify that a change of n_{H}(T) for the un-annealed first material is reversible over the temperature interval [Tₘᵢₙ, Tₘₐₓ]. A rate of change of the high complex refractive index with temperature dn_{H}(T)/dT represents a slope of the temperature dependence n_{H}(T) of the high complex refractive index (or, equivalently, a first derivative of curve 402.) A value of the real part of the high complex refractive index n*_{H}(Tₒₚ) for the un-annealed first material at an operational temperature Tₒₚ is specified as the coordinate of a point where a normal through Tₒₚ intersects curve 402.

FIG. 4B shows a graph 430 in which a temperature dependence n_{L}(T) of real part of the low complex refractive index of a second material—from which remaining of the layers of the ICEs are formed—is represented as curve 432 for temperatures much lower than the annealing temperature T_{AL} of the second material, Tₘₐₓ<<T_{AL}. The arrows at both ends of curve 432 signify that a change of n_{L}(T) for the un-annealed second material is reversible over the temperature interval [Tₘᵢₙ, Tₘₐₓ]. A rate of change of the low complex refractive index with temperature dn_{L}(T)/dT represents a slope of the temperature dependence n_{L}(T) of the low complex refractive index (or a first derivative of curve 432.) A value of the real part of the low complex refractive index n*_{L}(Tₒₚ) for the un-annealed second material at an operational temperature Tₒₚ is specified as the coordinate of a point where a normal through Tₒₚ intersects curve 432. Although not explicitly shown herein, temperature dependencies of imaginary parts of the high and low complex refractive indices of the first and second materials—from which adjacent layers of the ICEs are formed—can be represented in graphs similar to the graphs 400 and 430 and are available to the computer system 305. Additionally, a temperature dependence n_{S}(T) of the real component of a complex refractive index of a material of the substrate can be represented in a graph similar to the graphs 400 and 430 and is available to the computer system 305.

A temperature dependence of SECₜ(T) representing a measure of performance degradation for an un-annealed ICE—if the un-annealed ICEs were operated over the temperature interval [Tₘᵢₙ, Tₘₐₓ]—can be predicted based, at least in part, on the temperature dependence n_{H}(T), n_{L}(T) of the complex refractive indices shown in FIGS. 4A-4B and the target thicknesses t(1), . . . , t(N) of layers L(1), . . . , L(N) specified in the ICE design. FIG. 4C shows a graph 460 in which SECₜ(T) is represented as curve 462 over temperatures much lower than the annealing temperature range [T_{AL}, T_{AH}] of the ICE, Tₘₐₓ<<T_{AL}. The arrows at both ends of curve 462 signify that the temperature dependence of the SECₜ(T) of un-annealed ICEs is reversible. Here, SECₜ(T) is caused by a temperature dependence of deviations of the complex refractive indices n*_{H}(T), n*_{L}(T) of the layers of the un-annealed ICEs from their respective target complex refractive indices n*_{H}(Tₒₚ), n*_{L}(Tₒₚ) specified by the ICE design. A rate of change of the SECₜ(T) of un-annealed ICEs with temperature dSECₜ(T)/dT represents a slope of SECₜ(T) (or a first derivative of curve 462.) As expected, a minimum of SECₜ(T) (corresponding to maximum performance) for the un-annealed ICEs is obtained for a temperature about equal to the operational temperature Tₒₚ. In the vicinity of Tₒₚ, a slope of curve 462 is approximately zero. Additionally, an overall curvature of SECt(T) is mostly negative (or, equivalently, a derivative of dSECt(T)/dT is negative.) The temperature dependence of the SECₜ(T) of un-annealed ICEs and specification of maximum allowed SECₘₐₓ can be used to establish an operational temperature range ΔTₒₚ of the un-annealed ICEs to be fabricated in the following manner. A lower/upper bound of the operational temperature range ΔTₒₚ is a temperature smaller/larger than the operational temperature Tₒₚ where the maximum allowed SECₘₐₓ intersects curve 462. Note that the temperature dependence of the SECₜ(T) of un-annealed ICEs shown in FIG. 4C results in a narrow operational temperature range ΔTₒₚ for these un-annealed ICEs.

In this manner, the target fabrication temperature range ΔT_{fab} within which the temperature of the un-annealed ICEs will be maintained during fabrication is such that an upper bound of the target fabrication temperature range ΔT_{fab} is smaller than a lower bound T_{AL} of the annealing temperature range [T_{AL}, T_{AH}] of the ICEs. In the examples illustrated in FIGS. 4A-4B, the target fabrication temperature range ΔT_{fab} during fabrication of the un-annealed ICEs is centered on the operational temperature Tₒₚ. For instance, if ICEs with an annealing temperature range [T_{AL}, T_{AH}]=[245° C., 275° C.] were to be operated in an un-annealed state over an operational temperature interval ΔTₒₚ=[60° C., 90° C.] centered on an operational temperature Tₒₚ=75° C., then the target fabrication temperature range to be maintained during the fabrication of these un-annealed ICEs is set in accordance with one of the following examples.

FIG. 4D shows an example of a narrow fabrication temperature range ΔT_{fab}=[70° C., 80° C.] that is contained within the operational temperature range ΔTₒₚ. In some cases, T_{fab} coincides with Tₒₚ, such that the narrow fabrication temperature range ΔT_{fab} is centered on the operational temperature range ΔTₒₚ.

FIG. 4E shows an example of a broad fabrication temperature range ΔT_{fab}=[45° C., 105° C.] that encompasses the operational temperature range ΔTₒₚ. In some cases, T_{fab} coincides with Tₒₚ, such that the operational temperature range ΔTₒₚ is centered on the broad fabrication temperature range ΔT_{fab}.

FIG. 4F shows an example of a fabrication temperature range ΔT_{fab}=[105° C., 115° C.] that does not overlap and is above the operational temperature range ΔTₒₚ, such that a lower bound of the fabrication temperature range ΔT_{fab} is larger than an upper bound of the operational temperature range ΔTₒₚ. In these cases, T_{fab} also is larger than the upper bound of the operational temperature range ΔTₒₚ.

FIG. 4G shows an example of a fabrication temperature range ΔT_{fab}=[80° C., 115° C.] that overlaps and extends above the operational temperature range ΔTₒₚ. Here, an upper bound of the operational temperature range ΔTₒₚ is contained within the fabrication temperature range ΔT_{fab}. In these cases, T_{fab} can be smaller or larger than the upper bound of the operational temperature range ΔTₒₚ.

FIG. 4H shows an example of a fabrication temperature range ΔT_{fab}=[45° C., 70° C.] that overlaps and extends below the operational temperature range ΔTₒₚ. Here, a lower bound of the operational temperature range ΔTₒₚ is contained within the fabrication temperature range ΔT_{fab}. In these cases, T_{fab} can be smaller or larger than the lower bound of the operational temperature range ΔTₒₚ.

FIG. 4I shows an example of a fabrication temperature range ΔT_{fab}=[30° C., 45° C.] that does not overlap and is below the operational temperature range ΔTₒₚ, such that an upper bound of the fabrication temperature range ΔT_{fab} is smaller than a lower bound of the operational temperature range ΔTₒₚ. In these cases, T_{fab} also is smaller than the lower bound of the operational temperature range ΔTₒₚ.

### Example 2

In other implementations, ICEs are fabricated to be used in their annealed state, e.g., over a broad operational temperature range ΔTₒₚ, e.g., more than 50%, relative to its center value Tₒₚ, or at an operational temperature Tₒₚ comparable with the annealing temperature range. Annealed ICEs are exposed, at least during fabrication, at temperatures that exceed the lower bound of the annealing temperature range.

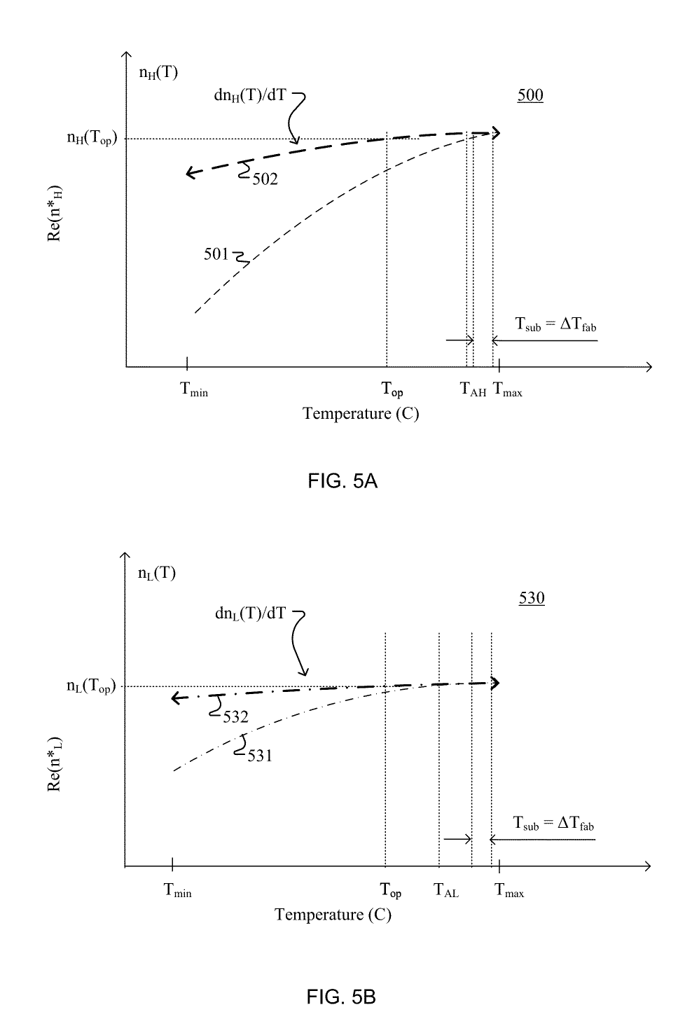

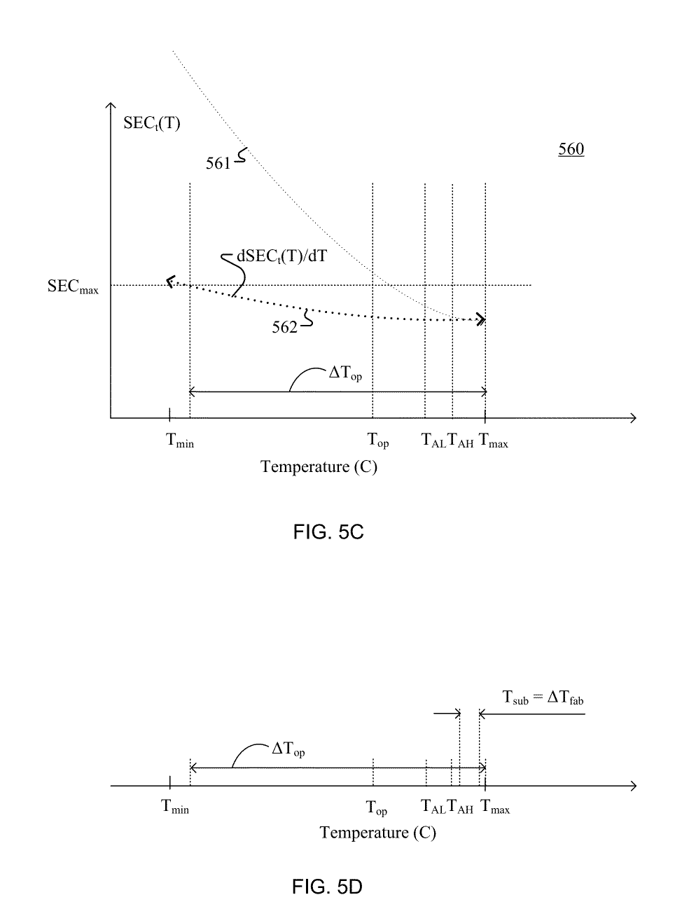

FIG. 5A shows a graph 500 in which a temperature dependence n_{H}(T) of real part of the high complex refractive index of a first material—from which some of the layers of the ICEs are formed—is represented as curves 501, 502 for temperatures that extend from below an annealing temperature T_{AH} of the first material to above this temperature, Tₘᵢₙ<T_{AH}<Tₘₐₓ. Curve 501 is the temperature dependence n_{H}(T) of the high complex refractive index as the un-annealed first material is heated for the first time from Tₘᵢₙ to Tₘₐₓ through the annealing temperature T_{AH}. An arrow at the high-temperature end of curve 501 and no arrow at the low-temperature end of it signify that the increase in n_{H}(T) is irreversible when the temperature of the un-annealed first material is raised from Tₘᵢₙ to Tₘₐₓ through T_{AH}. Curve 502 is the temperature dependence n_{H}(T) of the high complex refractive index of the annealed first material over the temperature interval [Tₘᵢₙ, Tₘₐₓ]. The arrows at both ends of curve 502 signify that a change of n_{H}(T) for the annealed first material is reversible over the temperature interval [Tₘᵢₙ, Tₘₐₓ]. A rate of change of the high complex refractive index with temperature dn_{H}(T)/dT represents a slope of the temperature dependence n_{H}(T) of the high complex refractive index (or a first derivative of curve 502.) A value of the real part of the high complex refractive index n*_{H}(Tₒₚ) for the annealed first material at an operational temperature Tₒₚ is specified as the coordinate of a point where a normal through Tₒₚ intersects curve 502.

FIG. 5B shows a graph 530 in which a temperature dependence n_{L}(T) of real part of the low complex refractive index of a second material—from which remaining of the layers of the ICEs are formed—is represented as curves 531, 532 for temperatures that extend from below an annealing temperature T_{AL} of the second material to above this temperature, Tₘᵢₙ<T_{AL}<Tₘₐₓ. Curve 531 is the temperature dependence n_{L}(T) of the low complex refractive index as the un-annealed second material is heated for the first time from Tₘᵢₙ to Tₘₐₓ through the annealing temperature T_{AL}. An arrow at the high-temperature end of curve 531 and no arrow at the low-temperature end of it signify that the increase in n_{L}(T) is irreversible when the temperature of the un-annealed second material is raised from Tₘᵢₙ to Tₘₐₓ through T_{AL}. Curve 532 is the temperature dependence n_{L}(T) of the low complex refractive index of the annealed second material over the temperature interval [Tₘᵢₙ, Tₘₐₓ]. The arrows at both ends of curve 502 signify that a change of n_{L}(T) for the annealed second material is reversible over the temperature interval [Tₘᵢₙ, Tₘₐₓ]. A rate of change of the low complex refractive index with temperature dn_{L}(T)/dT represents a slope of the temperature dependence n_{L}(T) of the low complex refractive index (or a first derivative of curve 532.) A value of the real part of the low complex refractive index n*_{L}(Tₒₚ) for the annealed second material at an operational temperature Tₒₚ is specified as the coordinate of a point where a normal through Tₒₚ intersects curve 532.

Note that the first and second materials of Example 2 may, but need not be, the same as the first and second materials described above in Example 1. For instance, if the first and second materials of Examples 1 and 2 are the same, than the temperature interval [Tₘᵢₙ, Tₘₐₓ] referenced in Example 2 extends to higher temperatures than the temperature interval [Tₘᵢₙ, Tₘₐₓ] referenced in Example 1. Alternatively, if the first and second materials of Example 2 have an annealing temperature interval [T_{AL}, T_{AH}] at lower temperatures than the annealing temperature interval [T_{AL}, T_{AH}] of the first and second materials of Example 1, than the temperature interval [Tₘᵢₙ, Tₘₐₓ] can be the same in the Examples 1 and 2.

A temperature dependence of SECₜ(T) representing a measure of performance degradation of an ICE—if the ICEs were operated over the temperature interval [Tₘᵢₙ, Tₘₐₓ]—can be predicted based, at least in part, on the temperature dependence n_{H}(T), n_{L}(T) of the complex refractive indices shown in FIGS. 5A-5B and the target thicknesses t(1), . . . , t(N) of layers L(1), . . . , L(N) specified in the ICE design. FIG. 5C shows a graph 560 in which SECₜ(T) is represented as curves 561, 562 over a temperature interval [Tₘᵢₙ, Tₘₐₓ] that includes the annealing temperature range [T_{AL}, T_{AH}] of the ICE. Here, SECₜ(T) is caused by a temperature dependence of deviations of the complex refractive indices n*_{H}(T), n*_{L}(T) of the layers of the ICE from their respective target complex refractive indices n*_{H}(Tₒₚ), n*_{L}(Tₒₚ) specified by the ICE design. Curve 561 is the temperature dependence of SECₜ(T) representing the performance degradation of un-annealed ICEs when the un-annealed ICEs are heated for the first time from Tₘᵢₙ to Tₘₐₓ through the annealing temperature range [T_{AL}, T_{AH}]. An arrow at the high-temperature end of curve 561 and no arrow at the low-temperature end of it signify that the decrease in SECₜ(T) is irreversible when the temperature of the un-annealed ICEs is raised from Tₘᵢₙ to Tₘₐₓ through [T_{AL}, T_{AH}]. Curve 562 is the temperature dependence of SECₜ(T) representing the performance degradation of the annealed ICEs over the temperature interval [Tₘᵢₙ, Tₘₐₓ]. The arrows at both ends of curve 562 signify that the temperature dependence of the SECₜ(T) of annealed ICEs is reversible. A rate of change of the SECₜ(T) of annealed ICEs with temperature dSECₜ(T)/dT represents a slope of SECₜ(T) (or a first derivative of curve 562.) As expected, a minimum of SECₜ(T) (corresponding to maximum performance) is obtained for a temperature about equal to the operational temperature Tₒₚ. However, in this example, a slope of curve 562 is approximately zero over a broad temperature range and not only in the vicinity of Tₒₚ. As described above, an operational temperature range ΔTₒₚ for the annealed ICE corresponds to temperatures for which SECₜ(T) does not exceed a maximum allowed SECₘₐₓ specified in the ICE design. Note that the temperature dependence of the SECₜ(T) of annealed ICEs shown in FIG. 5C results in a broad operational temperature range ΔTₒₚ for these annealed ICEs.

In this manner, the target fabrication temperature range ΔT_{fab} within which the temperature of the un-annealed ICEs will be maintained during fabrication is such that a lower bound of the target fabrication temperature range ΔT_{fab} is larger than a higher bound T_{AH} of the annealing temperature range [T_{AL}, T_{AH}] of the ICEs. In this manner, the annealing temperature range [T_{AL}, T_{AH}] of the ICEs is contained within the target fabrication temperature range ΔT_{fab}, to ensure that the constituent materials of the ICE are annealed during fabrication. As such, if ICEs with an annealing temperature range [T_{AL}, T_{AH}]=[145° C., 175° C.] were to be operated in an annealed state over a temperature range ΔTₒₚ=[25° C., 225° C.], then the target fabrication temperature range to be maintained during the fabrication of these annealed ICEs is set to ΔT_{fab}=[185° C., 215° C.], as shown in FIG. 5D. In these cases, T_{fab} also is larger than the upper bound of the annealing temperature range [T_{AL}, T_{AH}].

Referring again to FIGS. 3A-3C, the measurement system 304 associated with the ICE fabrication system 300 includes one or more instruments. For example, a physical thickness monitor (PM) (e.g., a quartz crystal microbalance) of the measurement system 304 is used to measure one or more deposition rates, R. The measured deposition rate(s) R is/are used to control power provided to the deposition source(s) 303 and its (their) arrangement relative to the current instances of ICEs 306 being fabricated at the target fabrication temperature Tfab to obtain a specified deposition rate R. For instance, if an ICE design specifies that a j^{th} layer L(j) of the N layers of an ICE is a Si layer with a target thickness t(j), a stack including the previously formed ICE layers L(1), L(2), . . . , L(j−1) is exposed to a Si source—from among the deposition sources 303—for a duration ΔT(j)=t(j)/R_{Si}, where the R_{Si} is a deposition rate of the Si source. The measured deposition rate(s) R and the times used to deposit the formed layers L(1), L(2), . . . , L(j−1), L(j) can be used by the computer system 305 to determine actual values of the thicknesses t′(1), t′(2), . . . , t′(j−1), t′(j) of these layers.

Actual values n*_{Si}(T_{fab}), n*_{SiO2}(T_{fab}) of complex refractive indices of materials of formed adjacent layers at the target fabrication temperature and thicknesses t′(1), t′(2), . . . , t′(j−1), t′(j) of the formed layers L(1), L(2), . . . , L(j−1), L(j) also are determined by measuring—with the measurement system 304—characteristics of probe-light that interacted with the formed layers. Note that probe-light represents any type of electromagnetic radiation having one or more probe wavelengths from an appropriate region of the electromagnetic spectrum. Throughout this specification, determining a complex refractive index n* of a layer means that both the real component Re(n*) and the imaginary component Im(n*) of the complex refractive index are being determined. The characteristics of the formed layers are measured with other instruments of the measurement system 304.

In some implementations, the measurement system 304 includes an ellipsometer used to measure, after forming the j^{th} layer of the ICEs 306, amplitude and phase components (Ψ(j), Δ(j)) of elliptically polarized probe-light—provided by an optical source (OS)—after reflection from the stack with j layers of ICEs that are being fabricated in the deposition chamber 301. In this case, the probe-light is provided by the source OS through a probe window of the deposition chamber 301 associated with the ellipsometer, and the reflected probe-light is collected by an optical detector (OD) through a detector window of the deposition chamber 301 associated with the ellipsometer. Here, the measured amplitude and phase components (Ψ(j), Δ(j)) are used by the computer system 305 to determine the (real and imaginary components of) complex refractive indices and thicknesses of each of the layers in the stack formed at the target fabrication temperature T_{fab}: n*_{Si}(T_{fab}), n*_{SiO2}(T_{fab}), t′(1), t′(2), . . . , t′(j−1), t′(j). The computer system 305 makes this determination by solving Maxwell's equations for propagating the interacted probe-light through the formed layers in the stack.

In other implementations, the measurement system 304 is a spectrometer used to measure, after forming the j^{th} layer of the ICE 306, a spectrum S(j;λ) of probe-light—provided by an optical source OS over a broad wavelength range [λₘᵢₙ, λₘₐₓ]—after reflection from (or transmission through—not illustrated in FIGS. 3A-3C) the stack with j layers of the ICEs that are being fabricated in the deposition chamber 301. In this case, the broad wavelength range source OS provides probe-light through a probe window of the deposition chamber 301 associated with the spectrometer, and an optical detector OD collects the reflected (or transmitted) probe-light through a detector window of the deposition chamber 301 associated with the spectrometer. Here, the measured spectrum S(j;λ) over the wavelength range [λₘᵢₙ, λₘₐₓ] is used by the computer system 305 to determine the (real and imaginary components of) complex refractive indices and thicknesses of each of the layers in the stack formed at the target fabrication temperature T_{fab}: n*_{Si}(T_{fab}), n*_{SiO2}(T_{fab}), t′(1), t′(2), . . . , t′(j−1), t′(j). The computer system 305 makes this determination by solving Maxwell's equations for propagating the interacted probe-light through the formed layers in the stack.

In some other implementations, the measurement system 304 is an optical monitor used to measure, after forming the j^{th} layer of the ICE 306, change of intensity I(j;λₖ) of probe-light—provided by an optical source (OS)—due to reflection from (or transmission through—not illustrated in FIGS. 3A-3C) the stack with j layers of the ICEs that are being fabricated in the deposition chamber 301. Here, the probe-light has one or more “discrete” wavelengths {λₖ, k=1, 2, . . . }. A discrete wavelength λₖ includes a center wavelength λₖ within a narrow bandwidth Δλₖ, e.g., ±5 nm or less; two or more wavelengths, λ₁ and λ₂, contained in the probe-light have respective bandwidths Δλ₁ and Δλ₂ that are not overlapping. The source OS can be a continuous wave (CW) laser, for instance. The optical monitor's source OS provides probe-light through a probe window of the deposition chamber 301 associated with the optical monitor, and an optical detector OD collects, through a detector window of the deposition chamber 301 associated with the optical monitor, the reflected (or transmitted) light with an intensity I(j;λₖ). Here, the measured change of intensity I(j;λₖ) is used by the computer system 305 to determine the (real and imaginary components of) complex refractive indices and thicknesses of each of the layers in the stack formed at the target fabrication temperature T_{fab}: n*_{Si}(T_{fab}), n*_{SiO2}(T_{fab}), t′(1), t′(2), . . . , t′(j−1), t′(j). The computer system 305 makes this determination by solving Maxwell's equations for propagating the interacted probe-light through the formed layers in the stack.

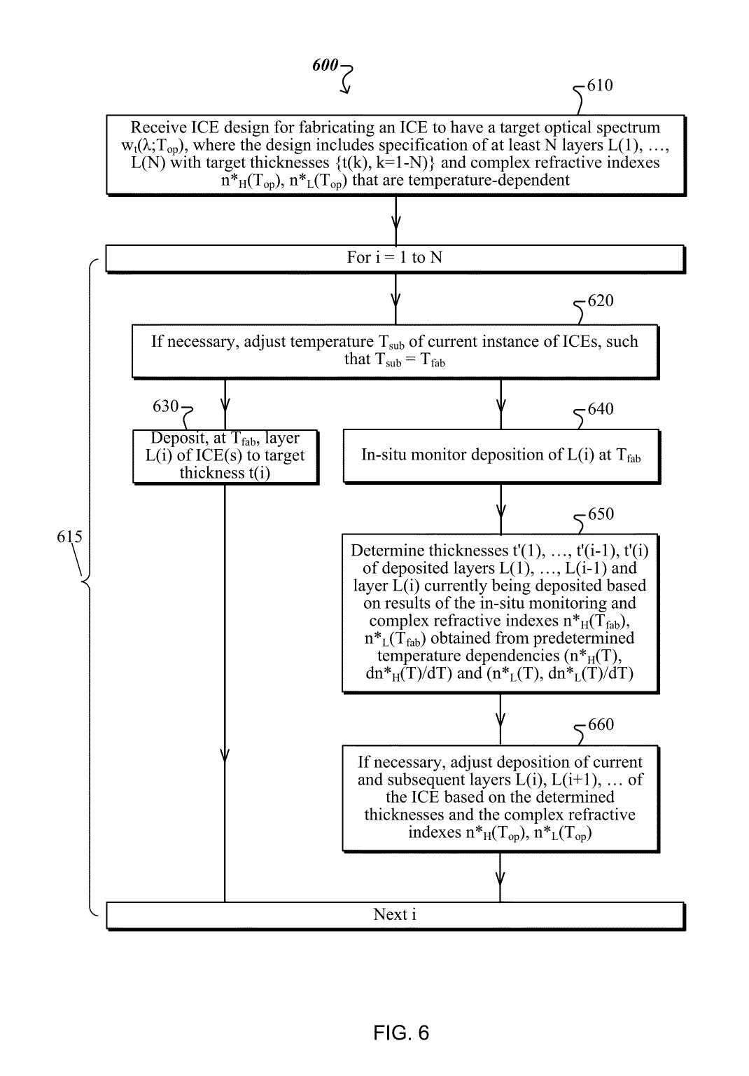

The computer system 305 includes one or more hardware processors and memory. The memory encodes instructions that, when executed by the one or more hardware processors, cause the fabrication system 300 to perform processes for fabricating the ICEs 306. Examples of such processes are described below in connection with FIG. 6. The computer system 305 also includes or is communicatively coupled with a storage system that stores one or more ICE designs 307, materials information 308 that includes predetermined temperature dependence of complex refractive indices and their respective rate of change, over a temperature interval [Tₘᵢₙ, Tₘₐₓ], e.g., given by curves 402, 432 or curves 502, 532. As described above in connection with FIGS. 4A-4C and 5A-5C, the temperature interval [Tₘᵢₙ, Tₘₐₓ] includes the target fabrication temperature range ΔT_{fab}, and optionally, it can include the operational temperature range ΔTₒₚ. For example, Tₘᵢₙ is an ambient temperature smaller than both ΔTₒₚ and ΔT_{fab}, and Tₘₐₓ is the maximum temperature of ΔT_{fab}. The foregoing materials information 308 can be used by the computer system 305 to control the heating source 310 for maintaining the temperature of the current instances of the ICEs 306 within a target fabrication temperature range ΔT_{fab} correlated with an operational temperature Tₒₚ, as described in Examples 1 and 2 above, or for adjusting deposition of a layer currently being deposited and of other layers remaining to be deposited.

The stored ICE designs can be organized in design libraries by a variety of criteria, such as ICE designs used to fabricate ICEs for determining values of a particular characteristic over many substances (e.g. the GOR ratio in crude oil, refined hydrocarbons, mud, etc.), or ICE designs used to fabricate ICEs for determining values of many properties of a given substance (e.g., viscosity, GOR, density, etc., of crude oil.) Additionally, the stored designs can be organized by operational temperature at which the fabricated ICEs will be used. For example, ICEs for determining the GOR ratio of wellbore fluids as part of a fixed-installation (e.g., like the one illustrated in FIG. 1A) at a first operational temperature corresponding to the ground surface 102, at a second operational temperature corresponding to a depth of 100 m under the ground surface, at a third operational temperature corresponding to a depth of 200 m under the ground surface, etc. As another example, ICEs for determining the GOR ratio of wellbore fluids as part of a wireline tool over a broad operational temperature range corresponding to temperature differences between two depth levels, e.g., between the ground surface 102 and a depth of 1000 m. In this manner, upon receipt of an instruction to fabricate an ICE for measuring a given characteristic of a substance at a specified operational temperature Tₒₚ or over a specified operational temperature interval ΔTₒₚ, the computer system 305 accesses such a design library and retrieves an appropriate ICE design 310 that is associated with the given characteristic of the substance at the specified Tₒₚ or over the specified ΔTₒₚ.

The retrieved ICE design 307 includes specification of a total number N of layers to be formed in the deposition chamber 301; specification of complex refractive indices n*_{H}(Tₒₚ) and n*_{L}(Tₒₚ) of first and second materials (e.g., Si and SiO₂)—corresponding to the operational temperature Tₒₚ—to form the N layers with adjacent layers having different complex refractive indices; and specification of target thicknesses {t(k), k=1−N} of the N layers. Implicitly or explicitly, the ICE design 307 also can include specification of a target optical spectrum wₜ(λ;Tₒₚ) associated with the given characteristic at Tₒₚ; and specification of a target SECₜ(Tₒₚ) representing expected performance degradation at Tₒₚ of an ICE associated with the retrieved ICE design 307. The foregoing items of the retrieved ICE design 307 were determined, prior to fabricating the ICEs 306, in accordance with the ICE design process 200 described above in connection with FIG. 2. In some implementations, the ICE design 307 can include indication of maximum allowed degradation SECₘₐₓ of the ICE caused by fabrication errors.

The complex refractive indices n*_{H}(Tₒₚ), n*_{L}(Tₒₚ) and target thicknesses {t(k), k=1−N)} of the N layers, as specified by the retrieved ICE design 307, are used by the computer system 305 to control deposition rate(s) of the deposition source(s) 303 and respective deposition times for forming the ICE layers, and the process parameters 315 are used by the computer system 305 to control temperature of the ICEs during the forming of the ICE layers. The temperature is controlled by the computer system 305 by monitoring whether a current instance of the ICEs' temperature matches a target fabrication temperature, and if not so, adjusting the current instance of the ICEs' temperature to match the target fabrication temperature using a heating source 310 (e.g., conductive heating source 310-A or radiative heating source 310-B, 310-C.) Also while forming the ICE layers, the computer system 305 instructs the measurement system 304 associated with the ICE fabrication system 300 to measure characteristics of probe-light that interacted with formed layers of ICEs being fabricated. The measured characteristics of the probe-light that interacted with the formed layers of the ICEs are used by the computer system 305 to determine complex refractive indices at the target fabrication temperature and thicknesses of the formed layers. If necessary, the computer system 305 also instructs the ICE fabrication system 300 to adjust the forming of layers remaining to be formed based on the determined complex refractive indices and thicknesses of the formed layers of the ICEs.

### (3.2) ICE Fabrication by In-Situ Controlling Temperature of ICEs

FIG. 6 is a flowchart of an example of an ICE fabrication process 600 for fabricating ICEs that allows for controlling temperature of the ICEs being fabricated. The process 600 can be implemented in conjunction with the ICE fabrication system 300 to fabricate ICEs to be used down-hole at elevated temperature, e.g., about 150° C., or over a wide temperature range, e.g., from about ambient temperature at the ground level to about 150° C. down-hole. In some cases, the fabricated ICEs will be operated at temperatures between −40° C. and 400° C. In such a context, the process 600 can be implemented as instructions encoded in the memory of the computer system 305, such that execution of the instructions, by the one or more hardware processors of the computer system 305, causes the ICE fabrication system 300 to perform the following operations.

At 610, an ICE design is received. The received ICE design includes specification of a substrate and N layers L(1), L(2), . . . , L(N), each having a different complex refractive index from its adjacent layers, and specification of complex refractive indices at an operational temperature Tₒₚ and target thicknesses t_{S}, t(1), t(2), . . . , t(N) of the substrate and the N layers. In this manner, an ICE fabricated in accordance with the received ICE design selectively weights, when operated at Tₒₚ, light in at least a portion of a wavelength range by differing amounts. The differing amounts weighted over the wavelength range correspond to a target optical spectrum wₜ(λ;Tₒₚ) of the ICE and are related to a characteristic of a sample at Tₒₚ. For example, a design process for determining the specified (1) substrate and number N of layers of the ICE, each having a different complex refractive index from its adjacent layers, and (2) complex refractive indices and thicknesses of the substrate and the N layers that correspond to the target optical spectrum wₜ(λ;Tₒₚ) of the ICE is described above in connection with FIG. 2. When fabricated ICEs are used in down-hole applications, the operational temperature Tₒₚ can be specified as a narrow operational temperature range ΔTₒₚ around a desired center value, e.g., ±5° C. around 150° C., or as a broad operational temperature range ΔTₒₚ, e.g., from 20° C. to 170° C. In other cases, the broad operational temperature range ΔTₒₚ can extend from −40° C. to 400° C. As described above in connection with FIGS. 4C and 5C, the operational temperature range ΔTₒₚ is a temperature interval over which degradation from ICE's performance due to temperature dependence of the complex refractive indices of the ICE is at most equal to a maximum allowed SECₘₐₓ of the ICE, where SECₘₐₓ represents degradation from a target ICE performance caused by fabrication errors. In this example, the target performance represents an accuracy with which the ICE predicts, when operated at Tₒₚ, known values of the characteristic corresponding to validation spectra of the sample taken at Tₒₚ. Here, predicted values of the characteristic are obtained when the validation spectra processed by the ICE are respectively integrated. In some implementations, the received ICE design also can include indication of the maximum allowed degradation SECₘₐₓ.

Loop 615 is used to fabricate one or more ICEs based on the received ICE design. Each iteration “i” of the loop 615 is used to form a layer L(i) of a total number N of layers. Here, the total number N of layers can be either specified in the received ICE design or updated during the ICE fabrication. Updates to the received ICE design are performed when necessary for preventing performance of the fabricated ICE to degrade under a threshold value.

At 620, a temperature of a current instance of the ICEs being fabricated is adjusted, if necessary, to a target fabrication temperature T_{fab}. In the example illustrated in FIGS. 3A-3C, a heating source 310 (e.g., electrical conductive elements 310-A included in a substrate support 302 in configuration 300-A of the ICE fabrication system, an IR laser or a black body emitter 310-B spaced apart from the substrate support 302 in configuration 300-B of the ICE fabrication system, or an inductive emitter 310-C adjacent the substrate support 302 in configuration 300-C of the ICE fabrication system) is used to maintain a temperature of substrates of the ICEs 306 being fabricated at the target fabrication temperature T_{fab}. The target fabrication temperature T_{fab} can be specified in terms of a target fabrication temperature range, ΔT_{fab}=[T_{fab}−δT, T_{fab}+δT], such that the temperature of the substrates of the ICEs 306 is maintained, during fabrication, within the target fabrication temperature range ΔT_{fab}.

In some implementations, when the ICEs to be fabricated will be operated in an un-annealed state at an operational temperature Tₒₚ lower than an annealing temperature range of the ICEs, an upper bound of the target fabrication temperature range ΔT_{fab} while forming the ICE layers is less than a lower bound of the annealing temperature range of the ICEs. The annealing temperature range of the ICEs is a temperature interval bound by respective annealing temperatures of constitutive materials of the ICEs. For example, the target fabrication temperature range ΔT_{fab} can be centered on the operational temperature Tₒₚ. Here, the target fabrication temperature range ΔT_{fab} can be contained within the operational temperature range ΔTₒₚ. Or, the target fabrication temperature range ΔT_{fab} can contain the operational temperature range ΔTₒₚ. As another example, at least an upper bound of the target fabrication temperature range ΔT_{fab} can be larger than the upper bound of the operational temperature range ΔTₒₚ. As yet another example, at least a lower bound of the target fabrication temperature range ΔT_{fab} can be lower than the lower bound of the operational temperature range ΔTₒₚ.

In other implementations, when the ICEs to be fabricated will be operated in an annealed state (at an operational temperature Tₒₚ lower than, included in or higher than an annealing temperature range of the ICEs), a lower bound of the target fabrication temperature range ΔT_{fab} exceeds an upper bound of the annealing temperature range of the ICEs. Here, the target fabrication temperature range ΔT_{fab} may be larger than the annealing temperature range by about 5, 10, or 20% of a value of T_{fab}, for instance.

At 630, the layer L(i) of the ICEs 306 is formed to a target thickness t(i) while a temperature of the current instance of the ICEs 306 is the target fabrication temperature T_{fab}. The target thickness t(i) of the layer L(i) can be specified by the received ICE design or updated based on optimization(s) carried out after forming previous one or more of the layers of the ICE. For some of the layers of the ICE, a deposition source having a deposition rate R is used for a total time duration ΔT(i)=t(i)/R to deposit the layer L(i) to its target thickness as part of a single deposition step. Other layers are deposited to the target thickness t(i) using multiple deposition steps by discretely or continuously forming respective sub-layers of the layer L(i). Here, the deposition rate used for depositing each of the sub-layers can be the same or different from each other. In the case when the deposition rates for forming the sub-layers are different, the last few sub-layers of the layer L(i) can be formed using slower rates than the ones used for forming the first few sub-layers of the layer L(i).

At 640, deposition of the layer L(i) is monitored in-situ. For instance, while the layer L(i) is formed, in-situ optical and/or physical measurements are performed to determine one or more one or more characteristics of the formed layer L(i). In the examples illustrated in FIGS. 3A-3C, the optical measurements performed using the measurement system 304 include at least one of (1) in-situ ellipsometry to measure amplitude and phase components {Ψ(i),Δ(i)} of probe-light interacted with a current instance of the ICE(s) being fabricated, (2) in-situ optical monitoring to measure change of intensity I(i;λₖ) of probe-light interacted with the current instance of the ICE(s) being fabricated, and (3) in-situ spectroscopy to measure a spectrum S(i;λ) of probe-light interacted with the current instance of the ICE(s) being fabricated. In-situ physical monitoring, e.g., with a crystal microbalance, is used to measure deposition rates, for instance.

For some of the layers of the received ICE design, the optical measurements can be skipped altogether. For some other layers, the optical measurements are carried out continuously during the deposition of a layer L(i), in some implementations. In other implementations, the optical measurements are taken a finite number of times during the deposition of the layer L(i). In the latter case, the finite number of times can represent times when at least some of sub-layers of the layer L(i) are completed.

At 650, complex refractive indices n*′_{H}(T_{fab}) and n′_{L}(T_{fab}) at T_{fab} and thicknesses t′(1), t′(2), . . . , t′(i−1), t′(i) of the layers L(1), L(2), . . . , L(i−1) formed in previous iterations of the loop 615 and the layer L(i) that is currently being formed are determined based only on the characteristics measured at 640. Alternatively, predetermined temperature dependencies n*_{H}(T), n*_{L}(T) and dn*_{H}(T)/dT, dn*_{L}(T)/dT of the complex refractive indices and their derivatives (or rates of change with temperature) are used to interpolate values of the complex refractive indices n*_{H}(T_{fab}) and n*_{L}(T_{fab}) at T_{fab}. Curves 402, 432 and 502, 532 are examples of such temperature dependencies described above in connection with FIGS. 4A-4B and 5A-5B. Here, the thicknesses t′(1), t′(2), . . . , t′(i−1), t′(i) of the layers L(1), L(2), . . . , L(i−1) formed in previous iterations of the loop 615 and the layer L(i) that is currently being formed are determined based on the characteristics measured at 640 and the interpolated values of the complex refractive indices n*_{H}(T_{fab}) and n*_{L}(T_{fab}) at T_{fab}. In some implementations, the values of the complex refractive indices n*′_{H}(T_{fab}) and n*′_{L}(T_{fab}) at T_{fab} determined from the characteristics measured at 640 and the values n*_{H}(Tfab) and n*_{L}(Tfab) at Tfab interpolated from the predetermined temperature dependencies n*_{H}(T), n*_{L}(T) and dn*_{H}(T)/dT, dn*_{L}(T)/dT are weighted to determine the complex refractive indices n*″_{H}(T_{fab}) and n*″_{L}(T_{fab}) at T_{fab} in the following manner:

n*″_{H}(T_{fab})=wₘₑₐₛ·n*′_{H}(T_{fab})+wᵢₙₜₑᵣ·n*_{H}(T_{fab}) (1)

n*″_{L}(T_{fab})=wₘₑₐₛ·n*′_{L}(T_{fab})+wᵢₙₜₑᵣ·n*_{L}(T_{fab}) (2)

In equations (1) and (2), a weight wₘₑₐₛ is used to weight the values of the complex refractive indices n′_{H}(T_{fab}) and n*′_{L}(T_{fab}) at T_{fab} determined from the characteristics measured at 640, and a weight wᵢₙₜₑᵣ is used to weight the values n*_{H}(T_{fab}) and n*_{L}(T_{fab}) at T_{fab} interpolated from the predetermined temperature dependencies n*_{H}(T), n*_{L}(T) and dn*_{H}(T)/dT, dn*_{L}(T)/dT. In some implementations, the weights wₘₑₐₛ and wᵢₙₜₑᵣ are about equal to each other, wₘₑₐₛ≈wᵢₙₜₑᵣ. In other implementations, the weight wₘₑₐₛ is greater than the weight wᵢₙₜₑᵣ, wₘₑₐₛ>wᵢₙₜₑᵣ, if an accuracy of measured characteristics of the probe-light exceeds a target accuracy, e.g., when multiple characteristics of the probe-light have been measured, e.g., through in-situ spectral ellipsometry, or through a combination of at least two in-situ ellipsometry, spectroscopy and optical monitoring measurements. In some other implementations, the weight wₘₑₐₛ is smaller than the weight wᵢₙₜₑᵣ, wₘₑₐₛ<wᵢₙₜₑᵣ, if the accuracy with which the characteristics of the probe-light have been measured fails to meet the accuracy target.

At 660, deposition of current and subsequent layers L(i), L(i+1), . . . of the ICE(s) is adjusted, if necessary, based on determined complex refractive indices and thicknesses t′(1), t′(2), . . . , t′(i−1), t′(i) of deposited layers L(1), L(2), . . . , L(i−1) and the layer L(i) currently being deposited. For example, complex refractive indices corresponding to the layer L(i) being currently formed and other layers L(i+1), L(i+2), . . . remaining to be formed can be adjusted based on (1) a comparison between values of the complex refractive indices and thicknesses of the layers of the current instance of the ICEs and their respective target values, and (2) the predetermined temperature dependencies n*_{H}(T), n*_{L}(T) and dn*_{H}(T)/dT, dn*_{L}(T)/dT. Here, if values of the determined complex refractive indices are smaller/greater than the respective target values n*_{H}(T_{fab}) and n*_{L}(T_{fab}) at T_{fab}, then the computer system 305 instructs the heating source 310 to increase/decrease the temperature of the instance of the ICEs being fabricated by an incremental temperature ∈ to a new target fabrication temperature T′_{fab}=T_{fab}+/−∈. The incremental temperature ∈ is determined by interpolating the predetermined temperature dependencies n*_{H}(T), n*_{L}(T) and dn*_{H}(T)/dT, dn*_{L}(T)/dT. Here, the comparison is performed using either the complex refractive indices n*′_{H}(T_{fab}) and n*′_{L}(T_{fab}) at T_{fab} determined from the characteristics measured at 640 or the weighted complex refractive indices n*″_{H}(T_{fab}) and n*″_{L}(T_{fab}) at T_{fab} determined in accordance with equations (1) and (2).

As another example, a deposition rate and/or time used to form the layer L(i) currently being formed and other layers L(i+1), L(i+2), . . . remaining to be formed can be adjusted based on a comparison between values of the complex refractive indices and thicknesses of the layers of the current instance of the ICEs and their respective target values. As yet another example, in order to determine whether target thicknesses of the layer L(i) being current formed and other layers L(i+1), L(i+2), . . . , L(N) remaining to be formed should be updated, the following verification can be performed.

An SEC(i;N;Tₒₚ) of the ICE is predicted to representing degradation in the ICE's performance at Tₒₚ if the ICE were completed to have the formed layers L(1), L(2), . . . , L(i−1) with the determined thicknesses t′(1), t′(2), . . . , t′(i−1), and the layer L(i) currently being formed and other layers L(i+1), L(i+2), . . . , L(N) remaining to be formed with target thicknesses t(i), t(i), . . . , t(N). Values of the complex refractive indices used for this prediction are either specified in the ICE design received at 610 or determined at 650 or a combination thereof. Here, the predicted SEC(i;N;Tₒₚ) is caused by deviations of the determined complex refractive indices and thicknesses of the formed layers from their respective complex refractive indices and target thicknesses specified by the current ICE design.

If the predicted SEC(i;N;Tₒₚ) at Tₒₚ does not exceed the maximum allowed SECₘₐₓ, SEC(i;N;Tₒₚ)≦SECₘₐₓ, then the forming of the current layer L(i) is completed in accordance to its target thickness t(i) and a next iteration of the loop 615 will be triggered to form the next layer L(i+1) to its target thickness t(i+1). If however, the predicted SEC(i;N;Tₒₚ) at Tₒₚ exceeds the maximum allowed SECₘₐₓ, SEC(i;N;Tₒₚ)>SECₘₐₓ, then target thicknesses of the layer L(i) currently being formed and other layers L(i+1), L(i+2), . . . , L(N) remaining to be formed are modified based on the determined complex refractive indices and thicknesses of the formed layers L(1), L(2), . . . , L(i). This optimization may change the total number of layers of the ICE from the specified total number N of layers to a new total number N′ of layers, but constrains the thicknesses of the layers L(1), L(2), . . . , L(i) (of the current instance of the ICE) to the determined thicknesses t′(1), t′(2), . . . , t′(i). In this manner, the optimization obtains, in analogy with the process 200 described above in connection with FIG. 2, new target thicknesses t″(i), t″(i+1), . . . , t″(N′) of the layer L(i) currently being formed and other layers L(i+1), . . . , L(N′) remaining to be formed, such that a new target SEC′ₜ(i;N′;Tₒₚ) of the ICE at Tₒₚ—for the ICE having the first layers L(1), L(2), . . . , L(i−1) formed with the determined thicknesses t′(1), t′(2), . . . , t′(i−1), and the layer L(i) currently being formed and other layers L(i+1), . . . , L(N′) remaining to be formed with the new target thicknesses t″(i), t″(i+1), . . . , t″(N′)—is minimum and does not exceed the maximum allowed SECₘₐₓ, SEC′ₜ(i;N′;Tₒₚ)≦SECₘₐₓ.

Once the previous instance of the ICE design is updated with specification of the new total number of layers N′ and the new target thicknesses t″(i), t″(i+1), . . . , t″(N′)—which are used to form the current layer L(i) and the remaining layers L(i+1), . . . , L(N′) and correspond to the new target SEC′ₜ(i;N′;Tₒₚ) at Tₒₚ—the forming of the current layer L(i) is completed in accordance with its new target thickness t″(i) and a next iteration of the loop 615 will be triggered to form the next layer L(i+1) from the new total number of layers N′ to its new target thickness t″(i+1). In this manner, the remaining layers of the ICE will be formed based on the updated ICE design, at least until another update is performed.

Some embodiments have been described in detail above, and various modifications are possible. While this specification contains many specifics, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

Other embodiments fall within the scope of the following claims.

1. A method comprising:
receiving, by a fabrication system, a design of an integrated computational element (ICE), the ICE design comprising specification of a substrate and a plurality of layers, their respective target thicknesses and complex refractive indices, wherein complex refractive indices of adjacent layers are different from each other, and wherein a notional ICE fabricated in accordance with the ICE design is related to a characteristic of a sample;
forming, by the fabrication system, at least some of the plurality of layers of an ICE in accordance with the ICE design; and
controlling, by the fabrication system during said forming, a temperature of the formed layers of the ICE such that the ICE, when completed, relates to the characteristic of the sample.

2. The method of claim 1, wherein
the completed ICE relates to the characteristic of the sample when operated at temperatures within an operational temperature range, and
said controlling comprises maintaining the temperature of the formed layers within a target fabrication temperature range.

3. The method of claim 2, wherein said maintaining comprises
monitoring whether a current instance of the temperature of the formed layers of the ICE is within the target fabrication temperature range, and if not so
adjusting the current instance of the temperature of the formed layers of the ICE to be within the target fabrication temperature range.

4. The method of claim 3, wherein said adjusting the current instance of the temperature of the formed layers of the ICE comprises heating a substrate support on which the formed layers of the ICE are disposed with electrical conductive heating elements distributed on the substrate support.

5. The method of claim 3, wherein said adjusting the current instance of the temperature of the formed layers of the ICE comprises heating a substrate support on which the formed layers of the ICE are disposed with a radiative heat source that is remote from the substrate support.

6. The method of claim 5, wherein the radiative heat source is a laser.

7. The method of claim 3, wherein said adjusting the current instance of the temperature of the formed layers of the ICE comprises heating a substrate support on which the formed layers of the ICE are disposed with an inductive heat source that is adjacent the substrate support.

8. The method of claim 2, wherein the operational temperature range is a temperature interval over which degradation from ICE's performance due to temperature dependence of the complex refractive indices of the ICE is at most equal to a maximum allowed degradation.

9. The method of claim 8, wherein the operational temperature range at which the ICE will be operated comprises −40 to 400° C.

10. The method of claim 2, wherein
an upper bound of the target fabrication temperature range during said forming of the ICE layers is less than a lower bound of an annealing temperature range of the ICE, and
the annealing temperature range of the ICE is a temperature interval bound by respective annealing temperatures of materials from which adjacent layers of the ICE are formed.

11. The method of claim 10, wherein the target fabrication temperature range is included within the operational temperature range of the ICE.

12. The method of claim 10, wherein an upper bound of the target fabrication temperature range is larger than an upper bound of the operational temperature range of the ICE, and a lower bound of the target fabrication temperature range is larger than a lower bound of the operational temperature range of the ICE.

13. The method of claim 12, wherein the lower bound of the target fabrication temperature range is larger than the upper bound of the operational temperature range of the ICE.

14. The method of claim 10, wherein a lower bound of the target fabrication temperature range is smaller than a smaller bound of the operational temperature range of the ICE, and an upper bound of the target fabrication temperature range is smaller than an upper bound of the operational temperature range of the ICE.

15. The method of claim 14, wherein the upper bound of the target fabrication temperature range is smaller than the lower bound of the operational temperature range of the ICE.

16. The method of claim 11, 12 or 14 wherein a width of the target fabrication temperature range is about 30% of its center value.

17. The method of claim 10, wherein the target fabrication temperature range includes the operational temperature range of the ICE.

18. The method of claim 2, wherein
a lower bound of the target fabrication temperature range during said forming of the ICE layers is larger than an upper bound of an annealing temperature range of the ICE, and
the annealing temperature range of the ICE is a temperature interval bound by respective annealing temperatures of materials from which adjacent layers of the ICE are formed.

19. The method of claim 18, wherein a difference between the lower bound of the target fabrication temperature range during said forming of the ICE layers and the upper bound of the annealing temperature range is about 30% of a center value of the target fabrication temperature range.

20. The method of claim 2, further comprising
in-situ monitoring said forming of the ICE layers at the target fabrication temperature range; and
determining, by the fabrication system, thicknesses of the formed layers of the ICE using results of said in-situ monitoring and complex refractive indices of the formed layers at the target fabrication temperature range obtained from predetermined temperature dependence of the complex refractive indices and rate of change of the complex refractive indices with the temperature.

21. The method of claim 20, wherein said in-situ monitoring comprises performing in-situ ellipsometry to measure amplitude and phase components of probe-light that interacted with the formed layers of the ICE.

22. The method of claim 20, wherein said in-situ monitoring comprises performing in-situ optical monitoring to measure change of intensity of probe-light that interacted with the formed layers of the ICE.

23. The method of claim 20, wherein said in-situ monitoring comprises performing in-situ spectroscopy to measure a spectrum of probe-light that interacted with the formed layers of the ICE.

24. The method of claim 20, wherein said in-situ monitoring comprises performing in-situ physical monitoring.

25. The method of claim 20, wherein
complex refractive indices at the operational temperature range specified in the ICE design are obtained from the predetermined temperature dependence of the complex refractive indices and the rate of change of the complex refractive indices with the temperature, and
the method further comprises adjusting, by the fabrication system, said forming, at least in part, based on the determined thicknesses and the complex refractive indices at the operational temperature range.

26. The method of claim 25, wherein said adjusting of said forming comprises updating target thicknesses of the layers remaining to be formed.

27. The method of claim 25, wherein said adjusting comprises changing a total number of layers specified by the ICE design to a new total number of layers.

28. The method of claim 25, wherein said adjusting of said forming comprises updating a deposition rate and/or time used to form the layers remaining to be formed.

29. The method of claim 25, wherein said adjusting of said forming comprises modifying complex refractive indices corresponding to the layers remaining to be formed.

30. A system comprising:
a deposition chamber;
one or more deposition sources associated with the deposition chamber to provide materials from which layers of one or more integrated computational elements (ICEs) are formed;
one or more supports disposed inside the deposition chamber, at least partially, within a field of view of the one or more deposition sources to support the layers of the ICEs while the layers are formed;
one or more heating sources thermally coupled with the one or more supports to heat the layers of the ICEs supported thereon while the layers are formed;
a measurement system associated with the deposition chamber to measure one or more characteristics of the layers while the layers are formed; and
a computer system in communication with at least some of the one or more deposition sources, the one or more supports, the one or more heating sources and the measurement system, wherein the computer system comprises one or more hardware processors and non-transitory computer-readable medium encoding instructions that, when executed by the one or more hardware processors, cause the system to form the layers of the ICEs by performing operations comprising:
receiving a design of an integrated computational element (ICE), the ICE design comprising specification of a substrate and a plurality of layers, their respective target thicknesses and complex refractive indices, wherein complex refractive indices of adjacent layers are different from each other, and wherein a notional ICE fabricated in accordance with the ICE design is related to a characteristic of a sample;
forming at least some of the plurality of layers of an ICE in accordance with the ICE design; and
controlling, during said forming, a temperature of the formed layers of the ICE such that the ICE, when completed, relates to the characteristic of the sample.

31. The system of claim 30, wherein the one or more heating sources comprise a plurality of electrical conductive heating elements distributed on the one or more supports.

32. The system of claim 30, wherein the one or more heating sources comprise a radiative heat source that is disposed remotely from the one or more supports, such that at least one of the supports is at least partially within the field of view of the radiative heating source.

33. The system of claim 30, wherein the one or more heating sources comprise an inductive heat source that is disposed adjacently at least one of the supports.

34. The system of claim 30, wherein the measurement system comprises an ellipsometer.

35. The system of claim 30, wherein the measurement system comprises an optical monitor.

36. The system of claim 30, wherein the measurement system comprises a spectrometer.

37. The system of claim 30, wherein the measurement system comprises a physical monitor.
